(19) United States
(12) Patent Application Publication
(10) Pub. No.: US 2012/0122765 A1
(43) Pub. Date: May 17, 2012

(54) COMPOUNDS AND RELATED METHODS FOR MANIPULATING PARP-1-DEPENDENT CELL DEATH

(76) Inventors: Ted M. Dawson, Baltimore, MD (US); Valina L. Dawson, Baltimore, MD (US); Yingfei Wang, Baltimore, MD (US)

(21) Appl. No.: 13/294,938

(22) Filed: Nov. 11, 2011

Related U.S. Application Data
(60) Provisional application No. 61/412,419, filed on Nov. 11, 2010.

(51) Int. Cl. A61K 38/17; C12N 15/12; A61P 25/00; A61P 25/28; A61P 3/10; A61P 27/02; A61P 13/12; A61P 9/00; A61P 9/10; A61P 35/00; A61P 9/04; A61P 25/16; A61P 25/14; A61P 19/02; A61P 1/00; C12N 15/63; C12P 21/02; C12N 1/21; C12N 1/19; C12N 5/10; C12Q 1/68; C07K 14/435

(52) U.S. Cl. 514/1.9; 530/324; 530/326; 536/23.5; 514/17.7; 514/17.8; 514/17.9; 514/6.9; 514/20.8; 514/15.4; 514/16.4; 514/15.1; 514/19.3; 514/16.7; 435/320.1; 435/69.1; 435/252.33; 435/252.31; 435/252.34; 435/252.3; 435/254.2; 435/325; 435/348; 435/6.1

(57) ABSTRACT

Apoptosis inducing factor (“AIF”) contains a PAR-binding motif (“PBM”) that binds to Poly(ADP-ribose) (“PAR”). Binding of PAR to AIF via the PBM is required for AIF release from the mitochondria to occur, and that this PAR-related release is a key step in the programmed cell death process known as parthanatos, both in vitro and in vivo. Preventing or disrupting this release can inhibit parthanatos and thus be the basis for treatments for patients suffering from diseases or medical conditions during which parthanatos commonly occurs, including Parkinson's disease or diabetes, or patients who have had and are recovering from heart attack, stroke and other ischemia reperfusion-related injuries. Alternatively, agents could be identified that enhance the release of AIF, thereby promoting parthanatos and serving as potential anti-tumor chemotherapeutic agents.

### CROSS-REFERENCE TO RELATED APPLICATIONS

This application claims the benefit of priority to prior U.S. Provisional Patent Application No. 61/412,419, filed Nov. 11, 2010, the entirety of which is hereby incorporated by reference.

### STATEMENT REGARDING FEDERALLY SPONSORED RESEARCH OR DEVELOPMENT

This invention was made with government support under grant award numbers NS039148 and NS067525 awarded by the National Institutes of Health. The government has certain rights in the invention.

### FIELD OF THE INVENTION

The present invention relates to the genetic or pharmaceutical manipulation of PARP-1-dependent cell death. More particularly, the present invention relates to methods for treating a target patient by either inhibiting or causing parthanatos in target cells.

### BACKGROUND OF THE INVENTION

Poly(ADP-ribose) (“PAR”) polymerase-1 (“PARP-1”) is an important nuclear enzyme that responds to DNA damage and is required for DNA repair. Upon activation, PARP-1 catalyzes the transfer of ADP-ribose from nicotinamide adenine dinucleotide (“NAD⁺”) and conjugates PAR onto a variety of nuclear proteins such as histones, DNA polymerases, topoisomerases, and transcription factors as well as auto-modification of PARP-1 itself, thus regulating a variety of physiologic processes. Excessive activation of PARP-1 leads to an intrinsic cell death program, which has been designated parthanatos (or, alternatively, PARP-1-dependent cell death) to distinguish it from necrosis and apoptosis. Parthanatos is known to occur in many diseases and conditions such as stroke, Parkinson's disease, heart attack, diabetes, and ischemia reperfusion injury. PARP inhibition or PARP-1 gene deletion is markedly protective in models of many cell injury paradigms, including stroke, trauma, ischemia-reperfusion injury, diabetes, and neurodegenerative diseases, indicating that parthanatos plays a prominent role in these disorders.

The mitochondrial protein apoptosis-inducing factor (“AIF”) plays a pivotal role in parthanatos, during which AIF is released from the mitochondria and translocates to the nucleus. AIF is a mitochondrial oxidoreductase that, like cytochrome C, has two independent functions. The first is within mitochondria, involving cell survival, thought to be though assembly or stabilization of respiratory complex I. The second is as a promoter of parthanatos cell death. AIF is released into the cytoplasm following PARP-1 activation, ultimately entering the nucleus to induce cell death.

Prior investigations have found it difficult to separate AIF's dual functions because complete loss of AIF disrupts mitochondrial function and energy metabolism, thereby preventing an assessment of its role as a death effector (see D. Brown et al., Proc Natl Acad Sci USA, 2006). PAR acts as a pro-death signaling molecule in parthanatos. Following excessive PARP-1 activation, PAR, which is mainly produced in the nucleus, translocates to the cytosol and interacts with the mitochondrial outer surface where it induces AIF release. However, exactly how PAR stimulates the release of AIF is not known. Blocking mitochondrial AIF release or reducing AIF abundance has been found to protect cells against parthanatos, indicating that this AIF release plays a crucial role in cell death induced by PARP-1 activation. Calpain, a calcium-dependent intracellular cysteine protease, has been suggested to cleave AIF at the N-terminus and cause AIF release from mitochondria after transient focal ischemia. However, prior studies by Applicants and others have confirmed that calpain is not involved in mitochondrial AIF release during parthanatos. Decreasing PAR abundance by PAR glycohydrolase (“PARG”), which degrades the PAR polymer, prevents PARP-1-dependent AIF release and reduces parthanatos in N-methyl-D-aspartate (“NMDA”) receptor-mediated glutamate excitotoxicity and N-methyl-N-nitro-N-nitrosoguanidine (“MNNG”) toxicity, and markedly reduces infarct volume in mice after two hours of transient middle cerebral artery occlusion (“MCAO”) (see Y. Wang et al, Exp Neurol, 2009). Parthanatos therapeutics targeted at decreasing PAR, however, are not deemed promising because of the necessity of PAR to other critical functions in the cell. Knowledge regarding exactly how PAR interacts with AIF during parthanatos would create a new avenue for the development of therapeutics.

Thus, there remains a need in the art for methods and therapeutics for inhibiting parthanatos in a target group of cells.

### SUMMARY OF THE INVENTION

In light of the above needs, it is an object of one or mode embodiments of the present invention to provide methods for inhibiting parthanatos.

Furthermore, it is an object of one or more embodiments of the present invention to provide therapeutic methods for treating diseases or medical disorders by alleviating, stopping, and/or reversing a symptom associated therewith, where such diseases or disorders include stroke, trauma, ischemia-reperfusion injury, diabetes, and neurodegenerative diseases.

Additionally, it is an object of one or more embodiments of the present invention to provide target sequences for genetic manipulation to alleviate unwanted PARP-1-induced cell death in target cells of a patient.

While Applicants have previously suggested after performing a proteomic screen for PAR-binding proteins that AIF could be a candidate protein for PAR binding (see J. P. Gagne et al., Nucleic Acids Res., 2008), the actual interaction between PAR and AIF heretofore has been unproven and unexplained. Applicants have discovered that AIF contains a PAR-binding motif (“PBM”), that PAR binding to AIF is required for AIF release from the mitochondria to occur, and that this PAR-related release is key to PAR's ability to induce cell death in parthanatos, both in vitro and in vivo. This release from the mitochondria is essential to parthanatos, and preventing or disrupting this release can inhibit parthanatos. Further, Applicants have discovered that AIF binds PAR at a site distinct from where AIF binds DNA, and this interaction between AIF and PAR triggers AIF release from the cytosolic side of the mitochondrial outer membrane. Moreover, mutating the PAR-binding site in AIF allows the separation of AIF's role in energy metabolism from its cell death function, making it possible to leave the metabolic functions undisturbed while inhibiting parthanatos.

In particular, Applicants have confirmed through experiments that certain mutations of the PAR binding site in AIF does not affect its NADH oxidase activity, its ability to bind FAD or DNA, or its ability to induce nuclear condensation, but nonetheless significantly inhibits its binding with PAR. Such AIF mutants are not released from mitochondria and do not translocate to the nucleus or mediate cell death following PARP-1 activation. This mechanism by which PARP-1 initiates AIF-mediated cell death can thus be treated by genetic modification of the PBM, or by chemical binding or inhibition of the PBM while still leaving undisturbed AIF's bio-energetic cell survival-promoting functions.

Thus, Applicants have discovered a novel and useful mechanism for preventing cell death following activation of PARP-1 by administering agents that interfere with the PAR-AIF interaction. Such mechanisms would be useful in treating patients suffering from Parkinson's disease or diabetes, or patients who have had and are recovering from heart attack, stroke and other ischemia reperfusion-related injuries. In this regard, Applicants' identification of AIF as a PAR polymer-binding protein establishes that therapeutic compounds which inhibit the interaction of PAR polymer with AIF may be useful in mono or combination therapies as protective compounds against stressors which activate PARP-1. Alternatively, therapeutic agents could be developed which enhance the release of AIF in a target group of cells, thus serving as a potential therapeutic against cancerous and other tumors.

Embodiments of the invention thus can include methods for treating a disease and/or condition associated with unwanted parthanatos by administering an effective amount of a PAR-AIF binding inhibitor to a patient having one of the above diseases for the purpose of treating or alleviating parthanatos-related symptoms. Additionally, embodiments of the invention can include methods for treating any such diseases and/or conditions by genetically modifying the PBM of the sequence of AIF expressed by target cells of a patient to prevent or reduce the binding of PAR to such modified AIF in those target cells for the purpose of treating or alleviating parthanatos-related symptoms. Further, embodiments of the invention can include methods for identifying agents effective to treat parthanatos-related symptoms associated with any of the above diseases and/or conditions by testing prospective PAR-AIF binding inhibitors for their ability to bind to the PBM of AIF.

In this regard, a first embodiment of the present invention includes a compound comprising a peptide having a sequence comprising any one of SEQ. ID NO. 15 through SEQ. ID NO. 24 as defined herein, wherein X in the sequence listings represents a variable amino acid with the caveat that at least two X in the peptide is a hydrophobic amino acid selected from the group consisting of arginine (“A”) or leucine (“L”). Additional related embodiments include polynucleotide including an encoding region that encodes such compounds, recombinant vector s that include such polynucleotides, transformants having inserted therein such recombinant vectors, and methods for producing such compounds which include the step of culturing such transformants so as to induce the transformant to produce such compounds.

Further, a second embodiment of the present invention includes a method for treating a disease or condition presenting unwanted parthanatos in target cells, comprising causing said target cells to express a mutated version of AIF which does not bind with PAR.

Additionally, a third embodiment of the present invention includes a method for treating a disease or medical condition in a mammal where the disease or medical condition is known to cause unwanted parthanatos in certain target cells of the mammal. This method comprises administering to the mammal a pharmaceutically effective amount of a PAR-AIF binding inhibitor drug, wherein (1) the inhibitor drug following administration inhibits AIF from binding to PAR in said target cells substantially without interfering with non-PAR related cellular functions of AIF, or (2) the inhibitor drug prevents AIF from releasing from the mitochondria membrane in response to nuclear PAR release from the nucleus.

Also, a fourth embodiment of the present invention includes a method for screening drug candidates for potential efficacy in humans to treat symptoms caused by a disease or medical condition that causes unwanted parthanatos in target cells. This method comprises testing each drug candidate for the capability to prevent the release of AIF from the mitochondrial membrane in response to nuclear PAR release in the target cells.

Similarly, a fifth embodiment of the present invention includes a method for screening drug candidates for potential efficacy in humans to treat symptoms caused by a disease or medical condition that causes unwanted parthanatos in target cells. This method comprises testing each said drug candidate for the capability to inhibit the binding of AIF to PAR in the target cells.

The various embodiments of the invention having thus been generally described, several illustrative embodiments will hereafter be discussed with particular reference to several attached drawings and in view of various experimental examples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing of the processes in a cell illustrating the model established herein of PAR-dependent AIF release in parthanatos.

FIG. 1B is an array of three black and white photographs depicted representative gels obtained from overlay assays on recombinant AIF with affinity-purified biotin-labeled PAR.

FIG. 2A is an array of black and white photos of representative gels obtained from experiments examining the impact of MNNG upon HeLa cells; and FIG. 2B is a chart reporting the experimental data regarding obtained regarding the relative intensity of the interaction pre and post MNNG for these experiments.

FIG. 3A comprises a black and white photographs of representative gels obtained from co-immunoprecipitation of endogenous AIF with PAR polymer is post nuclear fractions; and

FIG. 3B is a chart summarizing the experimental data regarding relative intensity so obtained.

FIG. 4 is a color schematic diagram showing the similarities of portions of the sequences of AIF present in various different species and identifying sequences therein that may function as PAR-binding motifs.

FIG. 5 comprises black and white photos of representative dot-blot assays for full length WT-AIF, three AIF peptides formed from identified portions of mouse AIF, histone H3, and BSA in presence of ³²P-labeled PAR.

FIG. 6 comprises black and white photographs of representative gels from an overlay assay performed to examine the effect of mutating or deleting certain portions of mouse AIF.

FIG. 7A comprises a three-dimensional color drawing of a ribbon diagram for the full-length mouse AIF structure; FIG. 7B is a three-dimensional color drawing depicting an enlarged portion thereof in greater detail; and FIG. 7C is a three-dimensional color drawing depicting the surface and electrostatic potential of full-length mouse AIF.

FIG. 8A is a schematic chart depicting the variance between four mutated versions of mouse AIF prepared by Applicants and wild-type mouse AIF; FIG. 8B comprises a composite black and white photograph of three representative gels run in an overlay assay using these mutated versions; FIG. 8C is a chart reporting experimental results for relative the PAR binding so obtained; and FIG. 8D is a chart reporting experimental results for relative PAR binding of various mouse AIF mutant proteins.

FIG. 9 comprises a black and white photograph of EMSA gels showing representative results for wild-type mouse AIF and mutated AIF using ³²P-labeled PAR.

FIG. 10A comprises a composite black and white photograph depicting typical gels obtained from a pull-down assay for wild-type and modified AIF sequences; and FIG. 10B through FIG. 10D comprise charts reporting data obtained from experiments performed to asses the PAR polymer binding properties of the mutated mouse AIF short peptides formed from mutated versions of portions of mouse AIF.

FIG. 11A comprises a color drawing of a graph providing experimental results relating to the impact of His-WT-AIF, His-Pbm-AIF on NADH oxidase activity; FIG. 11B comprises a color drawing of a graph providing experimental results from spectrophotometric wave length scanning relating to the FAD binding properties of His-WT-AIF and His-Pbm-AIF; FIG. 11C comprises a black and white photograph of a representative gel for a DNA retardation assay; FIG. 11D comprises an array of color photographs showing the result of fluorescence microscopy for nuclei treated by TW-AIF and Pbm-AIF following DAPI staining; and FIG. 11E is a chart reporting a quantification of nuclei treated by WT-AIF and Pbm-AIF.

FIG. 12A is a black and white gel photograph of a representative gel run to measure PAR binding to AIF from postnuclear fractions via co-immunoprecipitation; FIG. 12B is a graph quantifying the results from five such assays; FIG. 12C comprises a black and white photograph of a representative gel for coimmunoprecipitation of PAR with WT-AIF-Flag in cortical neurons in the absence or presence of BDRE cocktail; and FIG. 12D comprises an array of 10 color fluorescence photographs.

FIG. 13A is a black and white photograph of a representative gel for an immunoblot analysis of nuclear and postnuclear fractions; and FIG. 13B comprises an array of 16 color fluorescence photographs.

FIG. 14 comprises a black and white photograph of a representative gel showing AIF release from mitochondria according to an experiment.

FIG. 15A and FIG. 15B are graphs reporting experimental results for experiments performed to characterize the kinetics of AIF-mitochondria binding.

FIG. 16 is a graph reporting experimental results for an immunoblot analysis performed to characterize AIF binding to mitochondria.

FIG. 17A and FIG. 17B are graphs reporting experimental results for to assess the susceptibility of cells expressing WT-AIF-Flag or Pbm-AIF-Flag to parthanatos following MNNG cytotoxicity.

FIG. 18A comprises an array of eight representative color fluorescence photographs showing the effect of WT-AIF-Flag or Pbm-AIF-Flag on NMDA-induced cytotoxicity in Harlequin mice cortical neurons; and FIG. 18B is a graph reporting the experimental results for cytotoxicity measured at 24 hours, 36 hours, and 48 hours after NMDA treatment.

FIG. 19A comprises color Nissl staining photographs representative of the lesions in mice following NMDA injection into the striatum; FIG. 19B is a graph reporting the relative average lesion volumes obtained; FIG. 19C comprises color photographs showing the expression of injected WT-AIF-Flag, Pbm-AIF-Flag, and GFP in the striatum; and FIG. 19D comprises representative color Nissl staining photographs of the striatum 60 hours after NMDA injection; and

FIG. 19E is a graph summarizing data regarding lesion volumes obtained from such experiments.

FIG. 20A comprises an array of color photographs showing representative results obtained for transduction of injected agents in Harlequin mouse brains; FIG. 20B is an array color Nissl staining photographs taken 60 hours post NMDA injection in transduced mice; and FIG. 20C is a graph of the experimental data obtained reporting the lesion volume percentages for each.

### DETAILED DESCRIPTION OF THE PREFERRED EMBODIMENTS

As used herein “effective amount” of a composition is an amount sufficient, as appropriate in context, to retard or prevent parthanatos, or to kill cells in, retard or prevent growth and/or metastases of a targeted tumor. Where appropriate in context, a “pharmaceutically effective amount” or a “therapeutically effective amount” of a composition is an amount that is sufficient when that amount is administered to a stricken animal as a pharmaceutical formulation or therapeutic, as appropriate in context, (1) to inhibit, retard or prevent unwanted parthanatos, or (2) to kill cells in, retard or prevent growth and/or metastases of a targeted tumor, or (3) to treat or relieve symptoms associated with a disease in which parthanatos is a prominent disease mechanism.

Polynucleotides of the invention include any polynucleotide having a nucleotide sequence that encodes the peptides of the invention, although DNA is preferred. Exemplary DNA includes genomic DNA, genomic DNA libraries, cellular or tissue cDNA, cellular or tissue cDNA libraries, and synthetic DNA. The vectors used in the libraries are not subject to any particular limitation, and may be, for example, bacteriophages, plasmids, cosmids or phagemids. Also, amplification may be carried out directly by a reverse transcription polymerase chain reaction (abbreviated below as “RT-PCR”) using total RNA or a mRNA fraction prepared from the above-mentioned cell or tissue.

Other hybridizable polynucleotides include, when calculations are done with a sequencing program such as FASTA or BLAST using the default parameters, DNA that is at least approximately 60%, at least approximately 65%, at least approximately 70%, at least approximately 75%, at least approximately 80%, at least approximately 85%, at least approximately 88%, at least approximately 90%, at least approximately 92%, at least approximately 95%, at least approximately 97%, at least approximately 98%, at least approximately 99%, at least approximately 99.3%, at least approximately 99.5%, at least approximately 99.7%, at least approximately 99.8%, or at least approximately 99.9% identical to polynucleotides encoding the subject amino acid sequence. The identity of an amino acid sequence or a nucleotide sequence can be determined using the above-described method.

Recombinant vectors of the invention include those that may be obtained by ligating (inserting) the polynucleotides of the invention to a suitable vector. More specifically, the recombinant vector may be obtained by cleaving purified polynucleotide (e.g., DNA) with a suitable restriction enzyme, then inserting the cleaved polynucleotide to a restriction enzyme site or multicloning site on a suitable vector, and ligating the polynucleotide to the vector. The vector for inserting the inventive polynucleotide is not subject to any particular limitation, provided it is capable of replication in the host. Vectors that may be used for this purpose include plasmids, bacteriophages, and animal viruses. Illustrative examples of suitable plasmids include plasmids from E. coli (e.g., pBR322, pBR325, pUC118 and pUC119), plasmids from Bacillus subtilis (e.g., pUB110 and pTP5), and plasmids from yeasts (e.g., YEp13, YEp24 and YCp50). An example of a suitable bacteriophage is the λ phage. Examples of suitable animal viruses include retroviruses, vaccinia viruses and insect viruses (e.g., baculoviruses).

Transformants of the invention include can those that may be created by introducing into a suitable host the recombinant vector, obtained as described above, which includes a polynucleotide of the invention (i.e., a polynucleotide encoding a peptide of the invention). The host is not subject to any particular limitation, provided it is capable of expressing the polynucleotide of the invention. Examples include bacteria of the genera Escherichia, Bacillus, Pseudomonas and Rhizobium, yeasts, animal cells and insect cells.

Introduction of the recombinant vector into the host and transformation thereby may be carried out by any of various commonly used methods. Examples of suitable methods for introducing the recombinant vector into the host cell include the calcium phosphate method (Virology, 52, 456-457 (1973)), lipofection (Proc. Natl. Acad. Sci. USA, 84, 7413 (1987)), and electroporation (EMBO J., 1, 841-845 (1982)). Examples of methods for transforming genus Escherichia bacteria include the methods described in Proc. Natl. Acad. Sci. USA, 69, 2110 (1972), and Gene, 17, 107 (1982). Methods for transforming genus Bacillus bacteria include the methods described in Molecular & General Genetics, 168, 111 (1979). Methods for transforming yeasts include the methods described in Proc. Natl. Acad. Sci. USA, 75, 1929 (1978). Methods for transforming animal cells include the methods described in Virology, 52, 456 (1973). Methods for transforming insect cells include the methods described in Bio/Technology, 6, 47-55 (1988). A transformant created by transformation with a recombinant vector containing the polynucleotide which codes for the peptide of the invention (i.e., the polynucleotides of the invention) may be obtained in this way.

One skilled in the art will understand that the subject peptides described herein may be produced in various known ways. These include by synthetic means or by culturing transformants of the invention under conditions that allow the polynucleotide encoding the peptide to be expressed, thereby inducing formation and accumulation of the inventive peptide, then isolating and purifying the peptide. It is well within the skill of one of ordinary skill in the art to select a mechanism for synthetically building a peptide, or to select the appropriate conditions for causing expression for a given transformant, and to implement suitable mechanisms for isolating and purifying the peptide. The transformant of the invention may be cultivated by a conventional method used for culturing hosts. In such cultivation, the peptide of the invention may be formed by the transformant and accumulates within the transformant or the culture broth, requiring the selection of suitable isolation and purification processes.

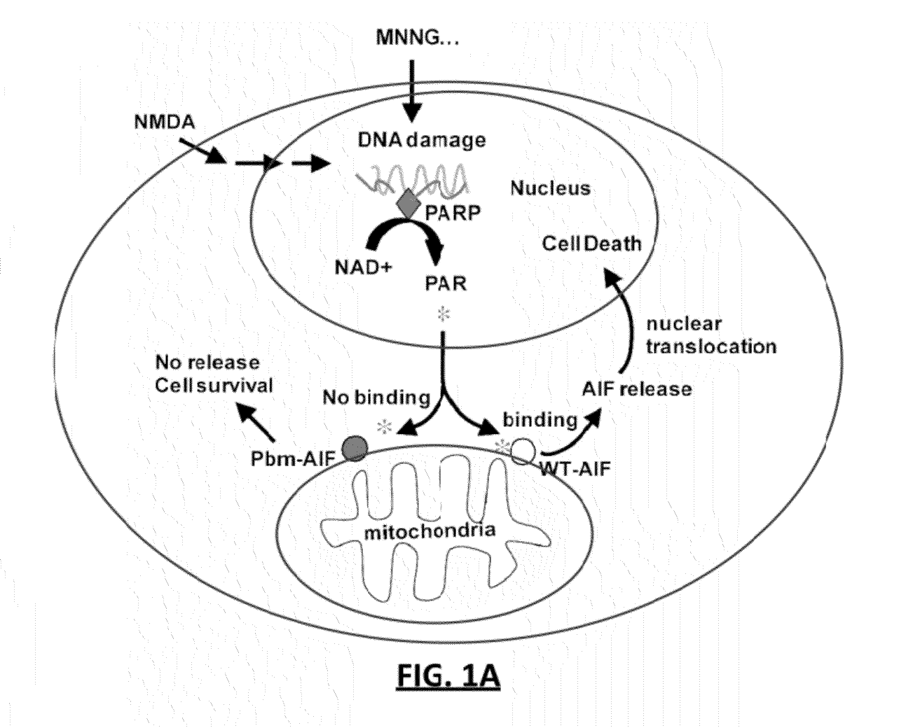

The various embodiments of the present invention relate to parthanatos, a key cell death mechanism involved in various diseases including ischemic injury and excitotoxicity. One pivotal step of parthanatos is that nuclear PAR signals to mitochondrial AIF and causes its release, thereby initiating a deadly interaction between these two organelles. Applicants have established via various experiments described herein that AIF is a PAR-binding protein and that PAR binding to AIF is critical for AIF release from the mitochondria following PARP-1 activation. FIG. 1A schematically depicts the model Applicants have constructed and experimentally confirmed for PAR-dependent AIF release in parthanatos in a single cell. The scheme shows that DNA damage induced by MNNG administration (or other alkylating agents, as indicated by the “ . . . ”) or NMDA excitotoxicity activates PARP-1, which in turn catalyzes PAR formation in the nucleus. PAR then translocates from the nucleus to the surrounding cytosol where it encounters mitochondria. How PAR exits the nucleus is not known with certainty, although it is possible that it may be carried out by a PARylated nuclear protein.

When PAR reaches the mitochondria, as depicted in FIG. 1A the PAR binds to a pool of AIF that is on the cytosolic side of the outer membrane of the mitochondria. PAR binding to AIF likely induces a conformation change in AIF that lowers its affinity for the mitochondrial outer membrane leading to its release, although the exact mechanism whereby PAR binding induces AIF release is unknown. At the mitochondria there are two pools of AIF. Approximately 80% of AIF is localized to the inner membrane and inner membrane space where it is protected from direct actions of PAR. However, approximately 20-30% of mitochondrial AIF is localized to the cytosolic side of the outer mitochondrial membrane. Applicants have confirmed by experiment that this binding to the AIF on the cytosolic side of the outer membrane induces release of the AIF. Thereafter, as shown in FIG. 1, this released AIF translocates to nucleus and therein causes cell death.

With reference to FIG. 1A, it will be apparent to one skilled in the art that there are various ways in which this parthanatos mechanism can be interfered with. For example, reducing PAR abundance with PARG expressed in the cytosol or interfering with PAR through neutralizing antibodies can reduce mitochondrial AIF release and subsequent cell death. Simply reducing the cellular concentration of PAR in this manner, however, it is an unfavored approach because, as noted above, PAR has a significant role in the creation of a variety of nuclear proteins such as histones. Similarly, directly reducing the amount of AIF in the cells is also a non-optimal approach for inhibiting parthanatos because of AIF's roles in respiration. However, Applicants have discovered that PAR binds specifically to a certain portion of AIF (which portion Applicants refer to herein as a “PAR binding motif” or “PBM”) that acts as the transducer that mediates AIF release from the mitochondria, which in turn allows AIF to translocate to the nucleus, and further discovered that this PBM is separate and apart from the portions of AIF involved in respiratory functions. Thus, as shown in FIG. 1A, natural PAR on the outer membrane of the mitochondria (which is labeled in FIG. 1 as “WT-PAR” for “wild-type PAR”) which bears a PBM will bind with PAR and release from the mitochondrial membrane. However, AIF which has been genetically modified to “delete” the PBM (of preferred embodiment of which is labeled in FIG. 1 as “Pbm-PAR” and defined below) remains on the membrane of the mitochondria and thus does not trigger cell death.

As detailed in the experiments set forth below, PAR bound to AIF saturably and with high affinity. However, Applicants have confirmed through experiment that if PAR fails to bind to AIF, such as by genetically modifying AIF or otherwise chemically inhibiting this binding, then AIF is not released from mitochondria subsequent to PARP-1 activation. Further, Applicants have found that the cells survive the toxic stimuli if AIF release is inhibited in this manner. Applicants thus identified PBMs in the various forms of AIF found in different species, and have identified similarities to the various PBMs to facilitate genetic modification.

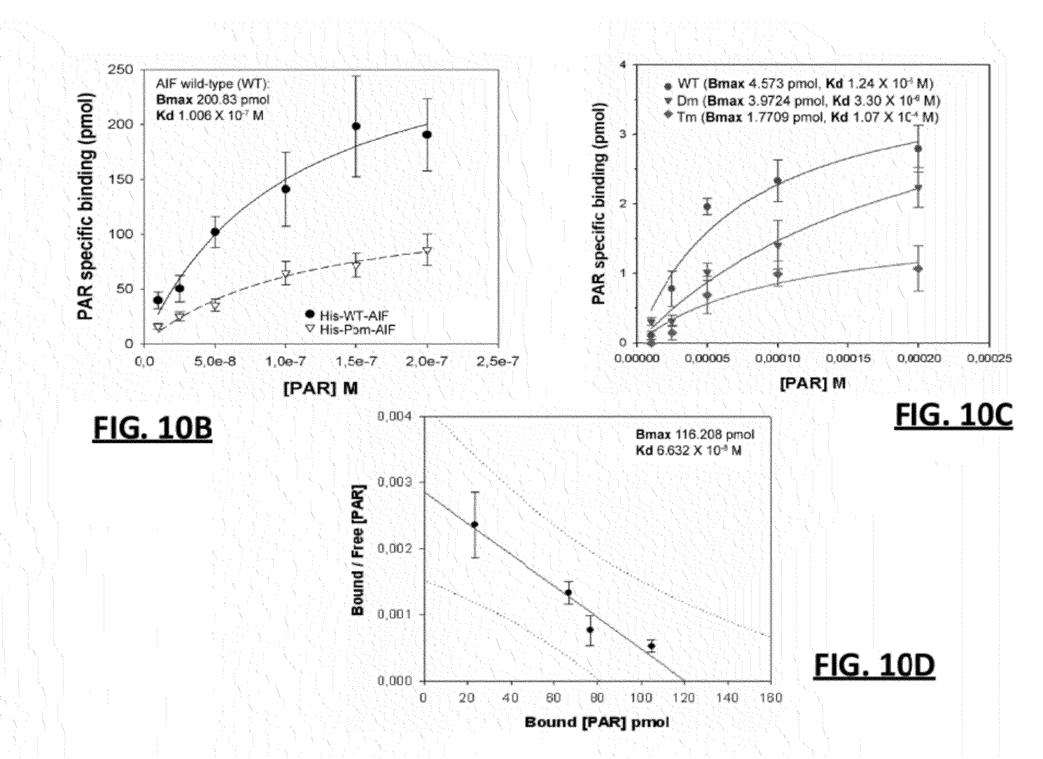

Amino acids 567-592 in the D3 domain of mouse AIF comprised the major PAR binding site in mouse AIF, as determined by PAR binding assays to peptides corresponding to the mouse AIF PAR-binding domain and mutational analysis of both these mimetic peptides and full length mouse AIF. The basic amino acids arginine 588, lysine 589, and lysine 592 were critical for PAR binding to mouse AIF. Their mutation to alanine or leucine markedly interfered with PAR binding to mouse AIF. Moreover, mutation of these amino acids to alanine prevented the release of mouse AIF from mitochondria following PAR treatment of purified mitochondria or PARP-1 activation with MNNG or NMDA. The failure of the Pbm-AIF to be released from the mitochondria following PARP-1 activation attenuated AIF-mediated cell death (FIG. 8). The PAR binding site is distinct from the AIF DNA binding site; thus, AIF appears to have evolved separate binding properties for DNA and PAR. Moreover, AIF's cell death functions can be separated from its role in mitochondrial respiration and cell survival.

PAR binds to various proteins, thereby affecting their physiologic function. PAR also appears to function as a scaffold, assembling PAR-binding proteins into a signaling complex that confers DNA damage-induced NF-κB activation. AIF is the first identified PAR-binding protein involved in cell death. How PAR-AIF binding modulates mitochondrial function remains unclear, as does the mechanism whereby AIF enters the nucleus and causes chromatinolysis and cell death.

In particular, identification of AIF as a PAR-binding protein opens up opportunities for the development of compounds that inhibit the interaction of PAR with AIF, thus potentially protecting against parthanatos. Alternatively, agents could be identified that enhance the release of AIF, thereby promoting parthanatos and serving as potential cancer chemotherapeutic agents.

Various aspects of the invention, including how to make and use the same, will be illustrated by the following various experimental examples.

Unless otherwise set forth herein, the preparation of the tested proteins, including protein expression and purification, in the examples below was done using the following materials and procedures. Mouse AIF cDNA was subcloned into 6×His-tagged pET28b vector (obtained from Novagen) or GST-tagged pGex-6P-1 vector (obtained from GE Health Care) by EcoR I and Xho I restriction sites. The protein was expressed and purified from E. coli using glutathione sepharose and metal-affinity chromatography, respectively. Flag-tagged WT-AIF and Pbm-AIF were subcloned into pCI vector (obtained from Promega) by Xho I and Sal I restriction sites. The various recombinant versions of AIF (and mutations thereof) described herein, either with His-tag or Flag-tag, was used as indicated below. To exclude interference from the tags, non-tagged recombinant AIF was also used for PAR-binding assays and other AIF biochemical determinations. Recombinant AIF was initially prepared from GST-tagged-AIF, purified by Glutathione Sepharose, with the GST-tag subsequently removed by precision protease. The various mutants of PAR and its PBMs as used in the various experiments below were constructed by PCR and verified by sequencing. The primers used in sequential point mutation to construct mono-point, di-point, triple point, and quadruple point mutations of PAR are summarized in Table 1 below.

TABLE 1

| AIF Mutant | Primer ID | Primer Sequence (5′ to 3′) |
|---|---|---|
| Single | S-R588A | CCGAATGCCAATTGCAGCGAAGATCATTAAGGACGGTG |
| (Sm) | AS-R588A | CACCGTCCTTAATGATCTTCGCTGCAATTGGCATTCGG |
| Di | S-K589A | GAATGCCAATTGCAGCGGCGATCATTAAGGACGGTG |
| (Dm) | AS-K589A | CACCGTCCTTAATGATCGCCGCTGCAATTGGCATTC |
| Triple | S-K592A | GCAGCGGCGATCATTGCGGACGGTGAGCAAC |
| (Tm) | AS-K592A | GTTGCTCACCGTCCGCAATGATCGCCGCTGC |
| Quadruple | S-R583A | GCTATGGAACGTCTTTAACGCAATGCCAATTGCAGCGG |
| (Qm) | AS-R583A | CCGCTGCAATTGGCATTGCGTTAAAGACGTTCCATAGC |
| AIFK254A | S-K254A | CTCAGATTACCTTTGAAGCGTGCTTGATTGCAACG |
| | AS-K254A | CGTTGCAATCAAGCACGCTTCAAAGGTAATCTGAG |
| AIFR264A | S-R264A | CGGGAGGCACTCCAGCAAGTCTGTCTGCCATC |
| | As-R264A | GATGGCAGACAGACTTGCTGGAGTGCCTCCCG |
| AIFR588L; | S-RK589L | CCGAATGCCAATTGCACTGCTGATCATTAAGGACGGTG |
| K589L | AS-RK589L | CACCGTCCTTAATGATCAGCAGTGCAATTGGCATTCGG |
| AIFR588L; | S-RK592L | CCGAATGCCAATTGCACTGAAGATCATTCTGGACGGTGAGCAACATGAAG |
| K592L | AS-RK592L | CTTCATGTTGCTCACCGTCCAGAATGATCTTCAGTGCAATTGGCATTCGG |
| AIFK589L; | S-KK592L | CCGAATGCCAATTGCAAGGCTGATCATTCTGGACGGTGAGCAACATGAAG |
| K592L | AS-KK592L | CTTCATGTTGCTCACCGTCCAGAATGATCAGCCTTGCAATTGGCATTCGG |
| AIFR588L; | S-RKK592L | CCGAATGCCAATTGCACTGCTGATCATTCTGGACGGTGAGCAACATGAAG |
| K589L;K592L | AS-RKK592L | CTTCATGTTGCTCACCGTCCAGAATGATCAGCAGTGCAATTGGCATTCGG |

Unless otherwise set forth herein, biotin- and ³²P-labeled automodified PARP-1 synthesis and PARP-free PAR preparation referenced in the examples below were performed using the following materials and procedures. Biotin- and ³²P-labeled automodified PARP-1 were synthesized as described previously described by V. Schreiber et al. (Nat Rev Mol Cell Biol, 2006) and G. Brochu et al. (Anal Biochem, 1994). Briefly, PARP-1 purified up to the DNA-cellulose step (600 U/mg) was incubated with biotin-labeled NAD⁺ and ³²P-labeled NAD⁺ for 2 minutes at 30° C. Thereafter a non-labeled and non-isotopic NAD⁺ was added to the reaction mixture, which was incubated for further 28 minutes at 30° C. The high specific activity biotin-labeled NAD⁺ and ³²P-labeled automodified PARP-1 (80 cpm/nmol) were precipitated as previously E. Afar et al. (Biochim Biophys Acta, 1999). Biotin-labeled, non-radioactive, and ³²P-labeled free PAR were prepared and purified on a DHBB column as described previously by C. Kiehlbauch et al. (Anal Biochem, 1993). Polymer size was assessed by 20% TBE-PAGE (90 mM Tris-borate pH 8.0, 2 mM EDTA) and HPLC chromatography using a DEAE-NPR column as described by Dawson et al. (J Neurosci, 1993). PAR has a mean length of 40 ADP-ribose residues as determined by HPLC methods and gel electrophoresis. The range of size of PAR in this mix is 6-mer through 100-mer ADP-ribose units.

Unless otherwise set forth herein, structural characterization of the proteins referenced in the examples below were performed using the following materials and procedures. Ribbon models of the murine AIF monomer (Protein Data Bank ID no.: 1GV4) were created using Rastop molecular visualization software. Molecular surface representation and electrostatic potential were calculated using Swiss-PBD software.

Unless otherwise set forth herein, all nitrocellulose PAR-binding assays and EMSA assays in the examples below were performed using the following materials and procedures. Synthetic peptides or purified proteins were diluted in TBS-T buffer (0.5 μg/μl) and loaded onto a nitrocellulose membrane (0.1 μm) using a dot blot manifold system (obtained from Life Technologies, of Gaithersburg, Md.). The membranes were washed once with TBS-T buffer and air dried followed by incubation with indicated concentrations of ³²P-labeled automodified PARP-1, ³²P-labeled PAR, or biotin-labeled PAR for 1 hour at room temperature. After washing, the membranes were analyzed by autoradiography on Bio-Max MR (available from Kodak), or probed with anti-biotin antibody and visualized with X-ray films by the SuperSignal West Pico Chemiluminescent Substrate (obtained from Pierce Protein Research Products, of Rockford, Ill.). Fluorescent Sypro Ruby protein staining was performed on the nitrocellulose membrane to demonstrate equal loading. For EMSA analysis, 50 ng of purified proteins (100 ng/ml) were incubated with ³²P-labeled PAR for 1 minute at room temperature. Thereafter, samples were resolved in 5% PAGE-gel. The gel was heat dried and developed using a Typhoon 9400 Imager (obtained from GE Health Care). PAR binding affinity was determined by nitrocellulose saturation binding experiments using the ligand binding program of Sigma plot (9.0 software-SYSTAT).

Unless otherwise set forth herein, all in vitro PAR-binding assays in the examples below were performed using the following materials and procedures. To begin, 300 nM of purified WT-AIF or Pbm-AIF (i.e., AIF mutated in its PAR binding region) were incubated with ³²P-labeled PAR for 10 minutes at room temperature. Thereafter, samples were incubated with AIF antibody-linked protein G slurry for 1 hour. Samples were washed two times with PBS and boiled with 1× Laemmli sample buffer (obtained from BIO-RAD). Each soluble fraction was resolved in 20% TBE-PAGE. The gel was heat dried and developed using the Typhoon 9400 Imager.

Unless otherwise set forth herein, the determination of NADH Oxidase Activity and FAD binding of AIF in the various examples below was performed using the following materials and procedures. AIF's NADH oxidase activity was determined as previously described by M. Miramar et al. (J Biol Chem, 2001). In brief, the NADH oxidase activity was measured at room temperature in substrate solution containing 250 μM NADH in air-saturated 50 mM Tris-HCl, pH 8.0. The decrease in absorbance at OD340 nm was monitored right after the addition of recombinant His-tagged AIF into the substrate solution. In situ redox activity of recombinant His-tagged AIF was performed as previously described by J. Gagne et al. (Nucleic Acids Res, 2008). The AIF protein was separated on a 10% native gel and then the gel was briefly washed in distilled water. The gel was equilibrated in 2,2′-Di-p-nitrophenyl-5-5′-diphenyl-3,3′ (3-3′-dimethoxy-4-4′difenilen)tetrazolium chloride (“NBT”) solution for 20 minutes in the dark followed by the addition of 1 mM NADH to NBT solution to reduce NBT. FAD binding to AIF was monitored at 13 μM concentration of His-tagged AIF by wave length scanning ranging from 250-800 nm using Beckman Coulter DU800 UV/Vis spectrophotometer.

Unless otherwise set forth herein, all DNA gel retardation assays referenced in the examples below were performed using the following materials and procedures. AIF binding-mediated DNA mobility retardation was performed as described by H. Cande et al. (Nat Struct Biol, 2002). Recombinant His-tagged AIF (10 μg or 25 μg) was incubated with 1 kb DNA molecular weight marker (obtained from Fermentas) for 30 minutes at room temperature and loaded on 2.5% agarose gel prestained with ethidium bromide (1 μg/ml). Mobility shift was visualized under the Alpha Innotech UV illuminator.

Unless otherwise set forth herein, the lentivirus construction and virus production referenced in the examples below were performed using the following materials and procedures. WT-AIF-Flag or Pbm-AIF-Flag was subcloned into a lentiviral cFugw vector by Age I and EcoR I restriction sites, and its expression was driven by human ubiquitin C (“hUBC”) promoter. The lentivirus was produced by transient transfection of the recombinant cFugw vector into 293T cells with three packaging vectors: pLP1, pLP2 and pVSV-G (1.3:1.5:1:1.5). The viral supernatants were collected at 48 hours and 72 hours after transfection and concentrated by ultracentrifuge for 3 hours at 50,000×g.

Unless otherwise set forth herein, cell cultures, transfection, lentiviral transduction and cytotoxicity referenced in the examples below were prepared using the following materials and procedures. MEF cells from wild type (“WT”) or Harlequin (“Hq”) mice and HeLa cells were cultured in Dulbecco's Modified Eagle's Medium (obtained from Invitrogen) and supplemented with 10% Fetal Bovine Serum (obtained from HyClone), 100 μg/ml streptomycin, and 100 U/ml penicillin (obtained from Invitrogen). Flag-tagged AIF (both WT and Pbm) was transfected using lipofectamine Plus (obtained from Invitrogen). Primary neuronal cultures from cortex were prepared as previously described by M. Gonzales-Zuleta et al. (J Neurosci, 1998). Briefly, the cortex was dissected and the cells dissociated by trituration in modified Eagle's medium (“MEM”), 20% horse serum, 30 mM glucose, and 2 mM L-glutamine following a 10 minute digestion in 0.027% trypsin/saline solution (obtained from Gibco BRL, Gaithersburg, Md.). The neurons were plated on 15 mm multi-well plates coated with polyomithine or on coverslips coated with polyomithine. Neurons were maintained in MEM, 10% horse serum, 30 mM glucose, and 2 mM L-glutamine in a 7% CO₂ humidified 37° C. incubator. The growth medium was refreshed twice per week. In mature cultures, neurons represented 70-90% of the total number of cells. At day in vitro (“DIV”) 7-9, neurons were infected by lentivirus carrying WT-AIF-Flag, Pbm-AIF-Flag, or GFP (1×10⁹ T.U./ml) for 72 hours. PARP-1 dependent cell death was induced by either MNNG in MEFs or HeLa cells or NMDA in neurons. HeLa cells or MEFs were exposed to MNNG (50 μM and 500 μM, respectively) for 15 minutes and neurons (DIV 10-14) were exposed to NMDA as described by S. Yu et al. (Science, 2002) and M. Gonzales-Zuleta et al. Neurons were washed with control salt solution (“CSS”), which contained (in mM): 120 NaCl, 5.4 KCl, 1.8 CaCl₂, 25 Tris-Cl, glucose, pH 7.4. The neurons then were exposed to 500 μM NMDA plus 10 μM glycine in CSS for 5 min, and then post-exposed in MEM, containing 10% horse serum, 30 mM glucose, and 2 mM L-glutamine for various times before fixation, immunocytochemical staining, and confocal laser scanning microscopy. Cell viability was determined the following day by unbiased objective computer-assisted cell counting after staining of all nuclei with 7 μM Hoechst 33342 (obtained from Invitrogen) and dead cell nuclei with 2 μM propidium iodide (Invitrogen). The numbers of total and dead cells were counted with the Axiovision 4.3 software (obtained from Carl Zeiss MicoImaging, Germany). At least three separate experiments using at least six separate wells were performed with a minimum of 15,000-20,000 neurons or cells counted per data point. For neuronal toxicity assessments, glial nuclei fluoresce at a different intensity than neuronal nuclei and were gated out. The percentage of cell death was determined as the ratio of live to dead cells as compared with the percentage of cell death in control wells to account for cell death attributed to mechanical stimulation of the cultures.

Unless otherwise set forth herein, pulldown, co-immunoprecipitation, and immunoblotting assays referenced in the examples below were performed using the following materials and procedures. For the pulldown assays, NeutrAvidin beads or biotin-labeled PAR-immobilized NeutrAvidin beads were incubated with 500 ng recombinant WT-AIF or Pbm-AIF, washed in lysis buffer, and eluted by boiling in sample buffer. For co-immunoprecipitation, the post-nuclear cell extracts, which is the fraction prepared from whole cell lysates after removing nuclear proteins, were isolated in hypotonic buffer as previously described by S. Andrabi et al., and then incubated overnight with an antibody against PAR in the presence of protein A/G Sepharose (obtained from Santa Cruz Biotechnology, of Santa Cruz, Calif.), followed by immunoblot analysis with mouse anti-Flag antibody (obtained from Sigma). The proteins were separated on denaturing polyacrylamide gel electrophoresis (SDS-PAGE) and transferred to nitrocellulose membrane. The membrane was blocked and incubated overnight with the 1 mg/ml primary antibody (mouse anti-Flag; rabbit anti-AIF; or rabbit anti-PAR 96-10) at 4° C., followed by donkey anti-mouse or goat anti-rabbit IgG conjugated to HRP for 1 hour at room temperature. After washing, the immune complexes were detected by the SuperSignal West Pico Chemiluminescent Substrate. The integrity of the nuclear and postnuclear subcellular fractions was determined by monitoring histone and MnSOD immunoreactivity, respectively.

Unless otherwise set forth herein, mitochondria isolation and in vitro AIF release assays as referenced in the examples below were performed using the following materials and procedures. Mitochondria were prepared from HeLa cells and cortical neurons as follows by harvesting cells, re-suspending them in 2 ml MIB buffer (10 mM HEPES, pH 7.4, 300 mM sucrose, 1 mM EGTA), and homogenizing by 60 strokes with B type Dounce homogenizer. Nuclei were pelleted at 750×g for 5 minutes and mitochondria were spun down at 3,000×g for 10 minutes. Isolated mitochondria were dissolved in a minimum volume of sucrose buffer (10 mM HEPES, pH 7.4, 300 mM sucrose) and protein concentration was adjusted to 2 mg/ml. The functional integrity of isolated mitochondria was verified by measuring the acceptor control ratio using a Clark type oxygen electrode (obtained from Hansatech). The ratio was determined by dividing the ADP (0.8 mM)-stimulated, state 3 respiration rate with the state 4 respiration rate in the presence of oligomycin (2.5 μg/ml) as described by B. Polster et al (J Biol Chem, 2001). Mitochondria preparations giving a ratio higher than 5 were used for the experiments. In vitro AIF release was carried out at room temperature for 30 minutes in assay buffer (20 mM HEPES, pH 7.4, 125 mM KCl, 2 mM K₂HPO₄, 4 mM MgCl₂, 5 mM succinate, 2 mM rotenone, 3 μM ATP and 1 μM ADP) in the presence of 100 nM PAR and 1 mg/ml isolated mitochondria. Proteins released into the assay buffer were saved by centrifugation at 12,000×g for 5 minutes.

Unless otherwise set forth herein, nuclear shrinkage assays as referenced in the examples below were performed using the following materials and procedures. Nuclei were obtained by purification from HeLa cells as described previously by A. Mandir et al. (J Neurosci, 2000). For the assay, purified nuclei were incubated with recombinant WT-AIF or Pbm-AIF (20 ng/ml) in the presence of ATP (2 mM), phosphocreatine (20 mM) and creatine kinase (50 μg/ml) for 90 minutes at 37° C. Nuclei were stained by DAPI and examined by fluorescence microscopy.

Unless otherwise set forth herein, immunocytochemistry, immunohistochemistry and confocal microscopy as referenced in the examples entailed the following materials and procedures. For immunocytochemistry, cells were fixed 2 hours after treatment with 4% paraformaldehyde, permeabilized with 0.05% Triton X-100, and blocked with 3% bovine serum albumin in PBS. AIF was visualized by 2 μg/ml mouse anti-Flag/Cyt AffiniPure Donkey Anti-Mouse IgG or rabbit anti-AIF/Cy3 AffiniPure Donkey Anti-Rabbit IgG. Immunohistochemistry was performed using the antibody against Flag. Immunofluorescence analysis thereafter was carried out by using a LSM510 confocal laser scanning microscope (obtained from Carl Zeiss Microlmaging) as described by H. Wang et al. (J. Neurosci, 2004).

Unless otherwise set forth herein, the injection of lentivirus and NMDA in Hq mouse striatum as referenced in the examples below was performed using the materials and procedures generally set forth previously by A. Madir et al. Briefly, 3-month-old Hq mice were anesthetized with pentobarbital (45 mg/kg, i.p.). 1 μl Lentivirus (5×1010 T.U./ml) carrying WT-AIF-Flag, Pbm-AIF-Flag, or GFP was injected into striatum on both sides of the brain (rostral, 0.4 mm; lateral+/−1.7 mm; ventral 3.5 mm from bregma), and 1 μl AAV2 (1×1013 T.U./ml) carrying WT-AIF-Flag, Pbm-AIF-Flag or GFP was injected into CA1 in hippocampi on both sides of the brain (posterior, 2.0 mm; lateral+/−1.5 mm; ventral 1.5 mm from bregma) using a stereotactic frame (Kopf) (0.02 ml/min). The needle was left in place for an additional 8 minutes after injection. One week after the first injection, 20 nmol NMDA or normal saline in 0.8 μl was injected into the right and left striatum (0.02 μl/min), respectively. The needle was left in place for an additional 8 minutes after injection. Lesion volumes were assessed at 48 hours, 60 hours, or 168 hours after NMDA administration. At the indicated time point after the second injection, the mice were deeply anesthetized, and then perfused with saline and 4% paraformaldehyde. After postfixation and freezing in 20% glycerol-PBS, brain sections (30 μm) were obtained for Nissl staining and immunohistochemistry analysis.

Unless otherwise set forth herein, all statistical evaluation was carried out by Student's t-test between two groups and by one-way analysis of variance (“ANOVA”) followed by post hoc comparisons with the Bonferroni test using GraphPrism software within multiple groups. Data are shown as mean±S.E.M., and p<0.05 was considered significant.

### Example 1

The following experiment was performed to confirm whether AIF in fact binds to PAR. Applicants performed 3 trials of an overlay assay on recombinant AIF with affinity-purified biotin-labeled PAR. Histone H3, which binds to PAR with high affinity, was included as a positive control and bovine serum albumin (“BSA”) was included as a negative control, as described by P. Chang et al. (Nat Cell Bio, 2005). FIG. 1B depicts three photos side by side for gels representative of all trials for these overlay assays. As shown in FIG. 1B, AIF bound to biotin-labeled PAR in a concentration-dependent manner (see the increasingly darker bands from left to right in each gel at approximately 70 kDa under AIF). It can also be seen that PAR polymer bound with AIF in a similar pattern to histone H3, while BSA failed to bind with PAR polymer. Furthermore, to confirm this result, an electrophoretic mobility shift assay (“EMSA”) was performed for AIFₘ using ³²P-labeled PAR and histone H1 as a positive control. AIFₘ was found to cause a shift of PAR polymer similar to that cause by histone H1, a known PAR-binding protein. Thus, Applicants took the results of these two tests as confirming that AIF binds to PAR in vitro.

### Example 2

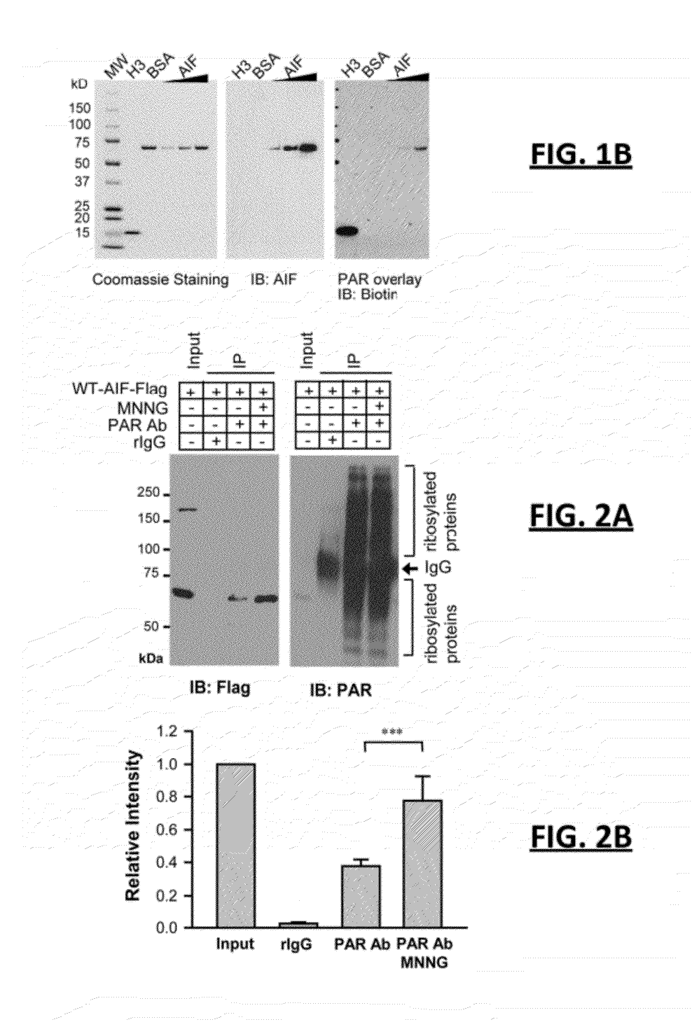

The following experiment was performed to confirm whether AIF in fact binds to PAR in intact cells. Applicants exposed HeLa cells stably transduced with lentivirus C-terminal Flag-tagged mouse wild type AIF (WT-AIF-Flag) to MNNG, a DNA alkylating agent that activates PARP-1 and kills cells primarily through parthanatos. PAR immunoprecipitation was performed from postnuclear fractions, which is the fraction prepared from whole cell lysates after removing nuclear proteins. FIG. 2A comprises two side by side black and white photos of representative gels obtained by Applicants, showing the impact of MNNG upon these HeLa cells (left photo being HeLa cells without MNNG, and right photo being HeLa cells 2 hours after MNNG treatment at 50 μM for 15 min). In the left gel of FIG. 2A, it can be seen that WT-AIF-Flag co-immunoprecipitated with PAR in resting cells. However, following MNNG treatment (the right photo of FIG. 2A), the interaction between AIF and PAR was significantly increased. Notably, WT-AIF-Flag did not co-immunoprecipitate with IgC, confirming the specificity of the interaction. FIG. 2B is a chart reporting the data regarding the relative intensity of the interaction pre and post MNNG for these experiments (*** indicates p<0.001).

### Example 3

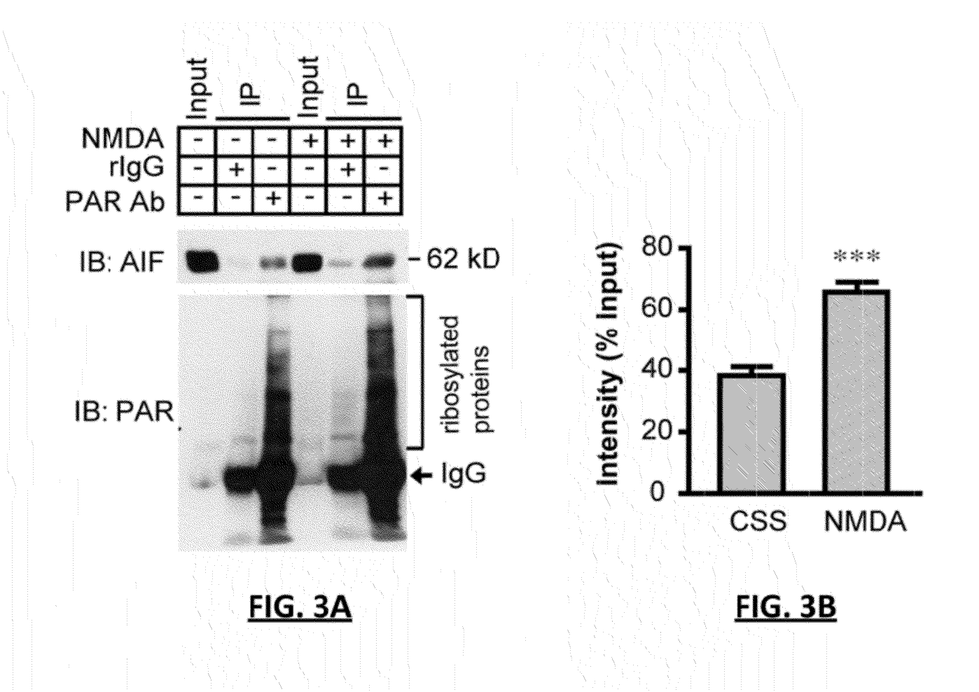

Next, to determine whether endogenous AIF interacts with PAR polymer, the interaction between endogenous PAR and AIF was explored in primary cortical neurons under both resting conditions and after NMDA glutamate receptor stimulation. This stimulation is known to activate PARP-1 potently and kill neurons through parthanatos. For this test, cortical neurons were homogenized and fractionated in CSS as described above, and FIG. 3A comprises a notated black and white photograph of a representative gel obtained from co-immunoprecipitation of endogenous AIF with PAR polymer is post nuclear fractions isolated from the cortical neurons 2 hours after NMDA treatment (500 μM for 5 minutes). Applicants found that endogenous AIF interacted with PAR in non-stimulated cortical neurons (see the first three columns of the gel photograph of FIG. 3A), but that interaction that was significantly increased following NMDA treatment (compare against the right three columns of the gel of FIG. 3A). FIG. 3B is a chart reporting the data regarding the relative intensity of the interaction in neurons resting (in CSS) and 2 hours post-MNNG treatment at 500 μM for 5 min (*** indicates p<0.001 by Student's t-test, n=6). Together, the data from the experiments of Examples 1-3 suggest that AIF is a PAR-binding protein and that PARP-1 activation increases the AIF-PAR interaction.

### Example 4

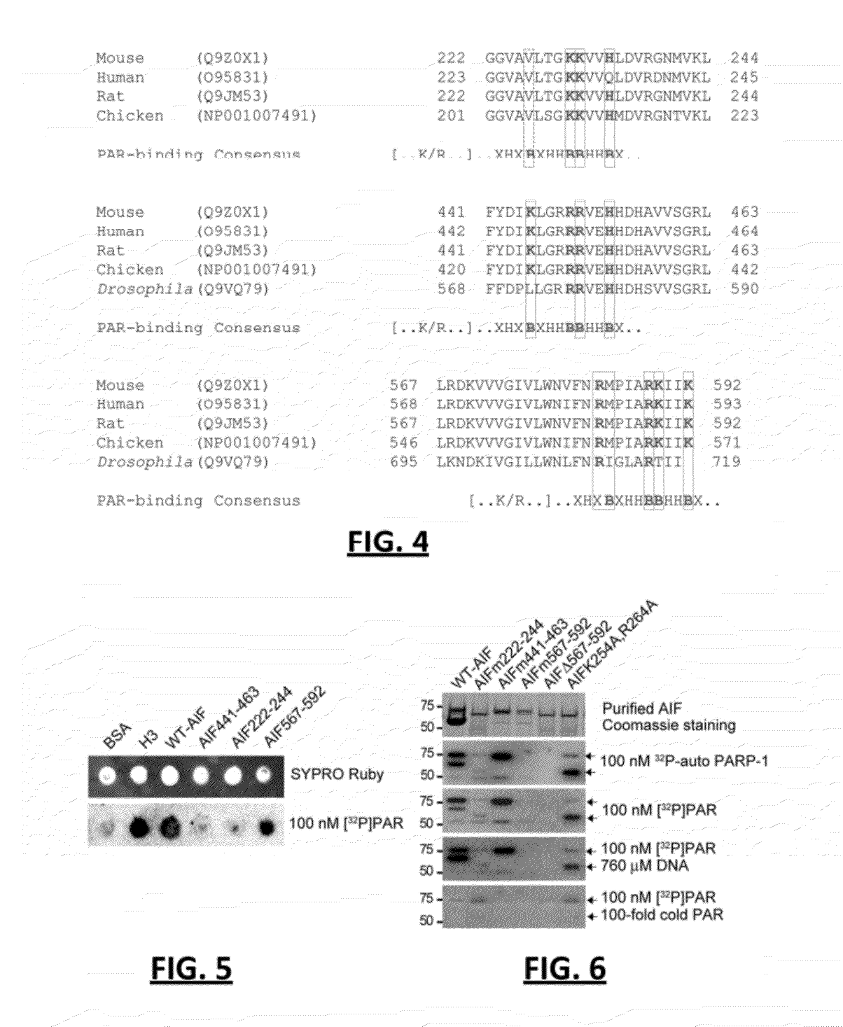

The PBM is believed to comprise approximately 20 amino acids and is characterized by hydrophobic amino acids separated by basic amino acids, and by a cluster of basic amino acid residues at the N-terminal side of the motif. To this end, to determine which domains of AIF are responsible for binding to PAR polymer, the amino acids sequence of AIF in various species of interest was evaluated in an effort to identify potential sequences comprising approximately 20 amino acids. The species examined included mouse (Accession Q9Z0X1), human (Accession Q95831), rat (Accession Q9JM53), chicken (Accession NP001007491), and Drosophila (Accession Q9VQ79). The results of this analysis are depicted in the schematic sequence comparison chart provided by FIG. 4. As shown in that figure, amino acids 222-244, 441-462, and 567-592 in mouse AIF were identified as being possible PAR-binding sites, and those putative PAR binding sites are conserved in the human, rat, and chicken (see the alignment of the putative binding sites in each species). In FIG. 4, the amino acid number is indicated on the right and left of the amino acid sequence, and species Accession numbers are in parentheses. As shown, the PAR-binding consensus sequence ([ . . . K/R . . . ] . . . XHXBXHHBBHHBX . . . ) comprises approximately 20 amino acids and is characterized by the presence of hydrophobic amino acids (H;ACGVILMFYW) spaced by basic amino acids (B; HKR) and by an accumulation of basic amino acid residues at the N-terminal site of the motif (KR), is indicated below each putative site. Conserved basic residues within the three putative PAR-binding sites are indicated in red bold letters. The 14 partial sequences of AIF from 5 species laid out in FIG. 4 correspond, from top to bottom, to sequences SEQ. ID NO. 1 through SEQ. ID NO. 14 as listed at the end of this application.

### Example 5

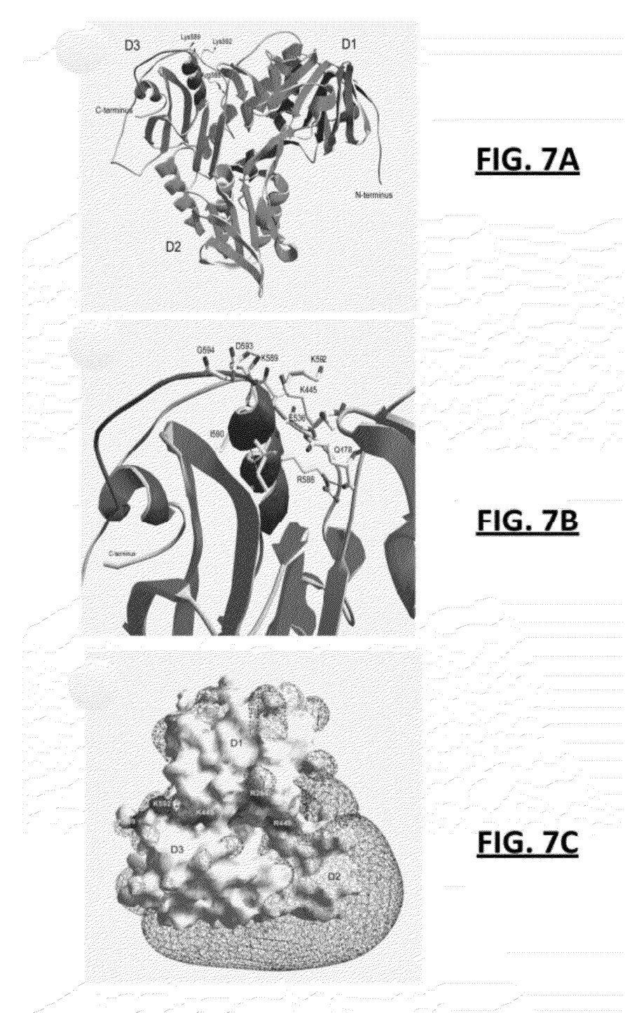

To determine whether these putative binding sites are in fact responsible for AIF binding to PAR, Applicants performed a dot-blot assay with peptides corresponding to these motifs in the presence of 100 nM purified ³²P-labeled PAR. FIG. 5 comprises black and white photos of representative dot-blot assay for full length WT-AIF, three AIF peptides formed from identified portions of AIFₘ, histone H3, and BSA in presence of 100 nM purified ³²P-labeled PAR.

A peptide composed of amino acids 567-592 of mouse AIF (“AIFₘ”), labeled AIF567-592 in FIG. 5) showed similar binding to PAR as achieved by full length wild type AIFₘ (“WT-AIF”) and histone H3. A peptide composed of AIFₘ amino acids 222-244 bound less well to ³²P-labeled PAR, and a peptide composed of AIFₘ amino acids 441-463 failed to bind PAR appreciably. The binding affinity of AIFₘ₅₆₇₋₅₉₂ peptide was thereafter determined by nitrocellulose saturation binding experiments using the ligand binding program of Sigma plot (9.0 software—SYSTAT) to be Kd of 1.24×10⁶M and Bmax of 4.55 pmol.

### Example 6

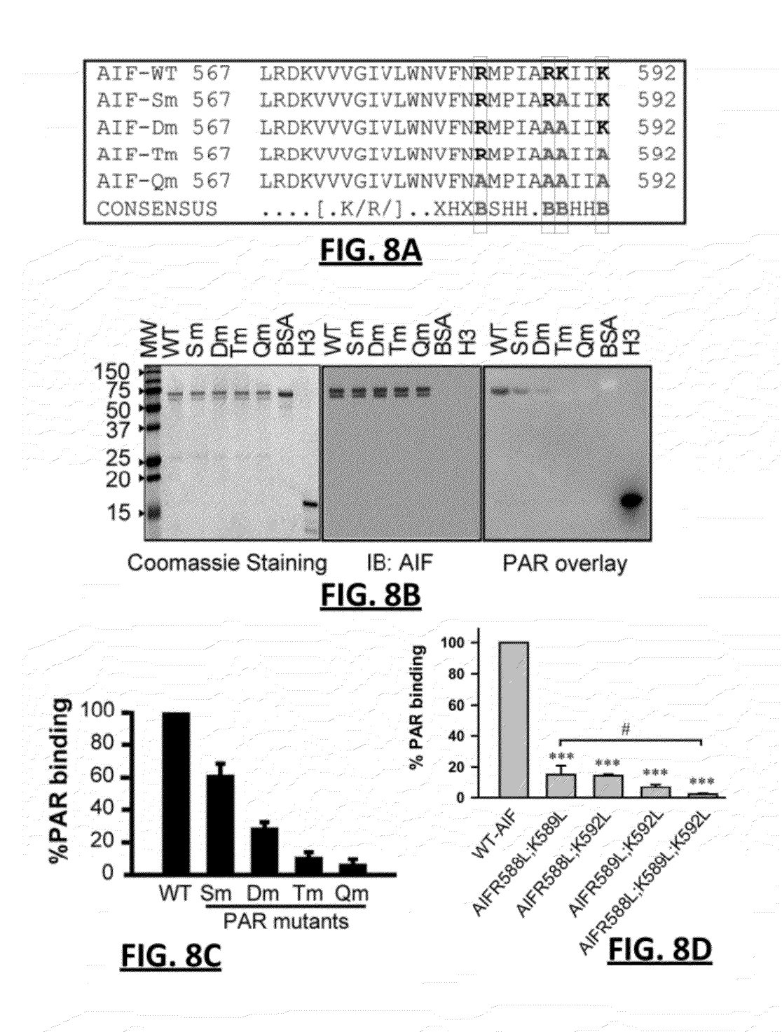

To further study these potential AIF PBMs, Applicants performed the following experiment to examine the effect of mutating AIFₘ amino acids 222-244, 441-463, and/or 567-592 to poly-alanine on the ability of AIF to bind PAR. FIG. 6 comprises black and white photographs of representative gels from an overlay assay of His-tagged WT-AIF, AIFₘ₂₂₂₋₂₄₄, AIFₘ₄₄₁₋₄₆₃, AIFₘ₅₆₇₋₅₉₂, AIFΔ567-592 (i.e., a mutant lacking AAs 567-592), and AIF(K254A, R264A) (i.e., mutating K to A at 254 and R to A at 264) mutants with ³²P-labeled automodified PARP-1 (equivalent to 100 nM of PAR), purified ³²P-labeled PAR (100 nM), or purified ³²P-labeled PAR with either a 100-fold excess of DNA or cold PAR. In particular, it was found that a His-tagged form of AIFₘ in which only amino acids 441 to 463 were mutated (AIFₘ₄₄₋₄₆₃) to ³²P-labeled automodified PARP-1 or purified ³²P-labeled PAR achieved comparable binding to that of unmutated WT-AIFₘ. The AIFₘ₂₂₂₋₂₄₄ mutant also bound PAR, but at a reduced abundance. Unlike AIFₘ₄₄₁₋₄₆₃ or AIFₘ₂₂₂₋₂₄₄, however, AIFₘ₅₆₇₋₅₉₂ failed to bind to ³²P-labeled automodified PARP-1 or purified ³²P-labeled PAR. The His-tagged AIF deletion mutant lacking amino acids 567-592 also failed to bind PAR, confirming that amino acids 567-592 contain the primary PAR-binding site in the intact mouse AIF protein. Together, these results led Applicants to conclude that amino acids 567-592 contain the major PBM in AIFₘ, but amino acids 222-244 in the mouse could also contribute to PAR binding to a lesser extent. The same can be assumed to corresponding sequences for other species, such as those identified in FIG. 4.

Additionally, to exclude the possibility that PAR polymer is binding to AIF through its DNA-binding domain, PAR polymer binding was assessed against the full length AIF DNA-binding mutant AIFK254A;R264A as described by H Cande et al. (Nat Struct Biol, 2002). Applicants found that the DNA-binding mutant AIFK254A; R264A still substantially binds to ³²P-labeled automodified PARP-1 and purified ³²P-labeled PAR polymer, as shown in the last column of the gel second from the bottom in FIG. 6. Moreover, Applicants found that a hundred fold excess of salmon sperm DNA fails to disrupt the PAR binding to WT-AIF, AIFₘ₄₄₁₋₄₆₃, AIFₘ₂₂₂₋₂₄₄ and AIFK254A;R264A, whereas cold PAR polymer disrupts PAR binding indicating that the PAR polymer-binding site is separable from the DNA-binding site of AIF.

Finally, Human AIF (“AIF_{H}”) is known to bind to DNA through a groove between the FAD-binding domain (“D1”) and C-terminal domain (“D3”). A mutant form of AIF_{H} in which lysine 255 is substituted with alanine and arginine 265 is substituted with alanine (AIFK255A;R265A) completely lacks DNA binding. To investigate the possible contribution of the AIF DNA-binding domain to PAR binding, Applicants assessed PAR binding to AIFₘ mutated in the same way (AIFK254A;R264A). As shown in FIG. 6, Applicants found that AIFK254A; R264A bound to ³²P-labeled automodified PARP-1 and purified ³²P-labeled PAR. Moreover, salmon sperm DNA at 760 μM failed to disrupt PAR binding to WT-AIF, AIFₘ₄₄₁₋₄₆₃, AIFₘ₂₂₂₋₂₄₄, or AIFK254A;R264A, whereas cold PAR blocked ³²P-labeled PAR binding. Thus, Applicants concluded that the AIF PAR-binding site is separable from its DNA-binding site, meaning that such binding could be inhibited by genetically modifying AIF without impacting the natural DNA binding capabilities of AIF or potentially by otherwise inhibiting the binding action of the PBMs.

### Example 7

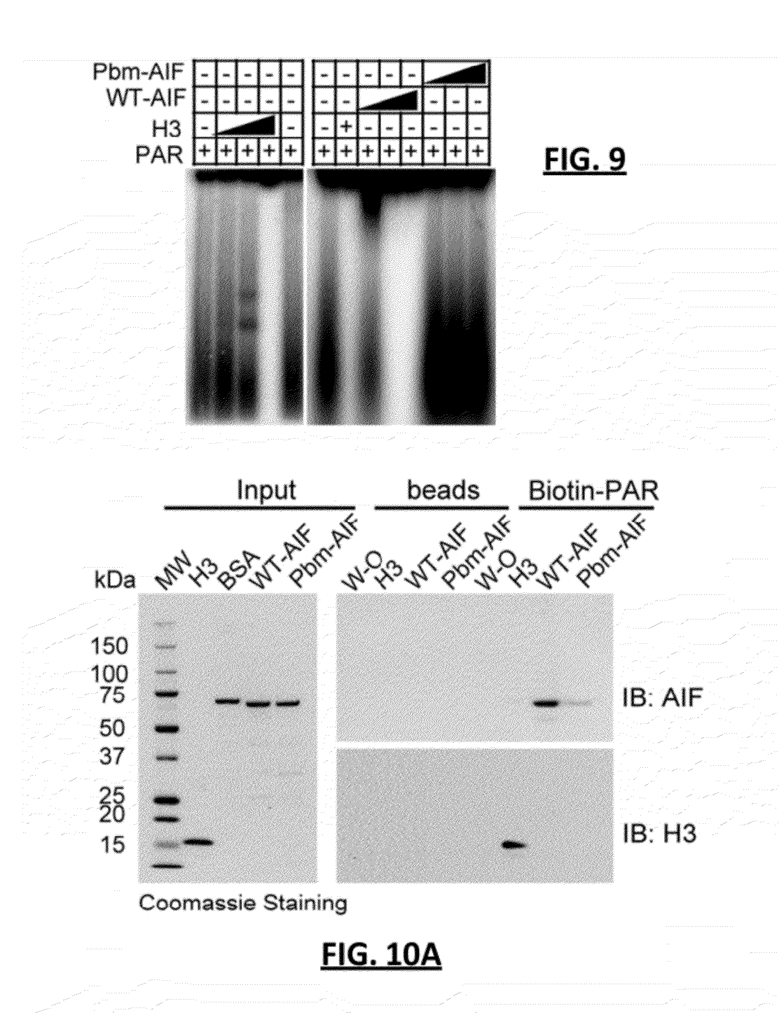

Since AIF has been crystallized, Applicants next modeled the AIFₘ PAR binding site. FIG. 7A comprises a three-dimensional color drawing of a ribbon diagram of the AIFₘ structure showing the three domains (D1 in blue, D2 in cyan and D3 in green) along with the basic helix-loop-helix domain constituting the PAR-binding domain (in red). Side chains of essential basic amino acids are indicated.

FIG. 7B comprises a close up of the three-dimensional color drawing of FIG. 7A, but this time labeling the structural details of the PAR-binding domain of AIF. In that drawing, stabilizing hydrogen bonds are shown in pink and for the labeled amino acids D is aspartic acid, E is glutamic acid, G is glycine, I is isoleucine, and Q is glutamine. Finally, FIG. 7C is a three-dimensional color drawing of AIFₘ showing the surface and electrostatic potential of AIFₘ. DNA binding sites are shown in blue and the PAR binding sites that do not overlap with the DNA binding sites are shown in red.

Analysis of the crystal structure of AIF as depicted in FIG. 7A through FIG. 7C revealed that amino acids 567-592 (the primary PAR-binding site Applicants identified) reside within an alpha helix and an external loop that is well suited to mediate the interaction between AIF and PAR. In particular, Applicants discovered that amino acids 567-592 are located in a predominantly hydrophobic cleft maintained in position through four hydrogen bonds. Several hydrogen bonds are involved in the interaction between side chains and backbone amine bonds inside the alpha helix located at the C-terminal domain of AIFₘ in the D3 domain. Further, a cluster of positively-charged basic amino acids (Arg⁵⁸³, Arg⁵⁸⁸, Lys⁵⁸⁹ and Lys⁵⁹²) potentially essential for PAR binding are located in the AIF D3 domain in close proximity to the DNA-binding domain, but distinct from it. Thus, the PAR-binding site does not overlap with the DNA binding site, and this result is consistent with the failure of the DNA binding mutant AIFK254A;R264A or excess DNA to affect PAR binding in the various experiments of Example 6.

### Example 8

Based on the above findings regarding the 3D structure and the PAR-binding consensus sequence, Applicants analyzed the effects of mutating basic amino acids in the AIFₘ PBM (i.e., within amino acids 567 and 592 of AIFₘ) likely to be critical for PAR binding. Various different mutations were performed according to the procedures described above, and then these different mutated versions of full length AIFₘ were tested comparatively for binding to PAR polymer. As detailed in the chart of FIG. 8A, Applicants produced four mutated versions of AIFₘ, namely AIF-Sm by substituting the arginine at 588 with alanine (essentially replacing SEQ. ID NO. 10 within wild-type AIFₙ, with SEQ. ID NO. 15), AIF-Dm by substituting both arginine at 588 and lysine at 589 with alanines (essentially replacing SEQ. ID NO. 10 within wild-type AIFₘ with SEQ. ID NO. 16), AIF-Tm substituting arginine at 588, lysine at 589, and at lysine 592 with alanines (essentially replacing SEQ. ID NO. 10 within wild-type AIFₘ with SEQ. ID NO. 17), and AIF-Qm by substituting arginine at 588, lysine at 589, lysine at 592, and at arginine 583 all with alanines (essentially replacing SEQ. ID NO. 10 within wild-type AIFₘ with SEQ. ID NO. 15). In FIG. 8A, X represents any amino acid, H represents any hydrophobic amino acid, and B represents any basic amino acid. Applicants thereafter performed three trials of a PAR overlay assay of full length WT-AIF and the four above-described mutant versions. The radioactive signal was quantified and normalized to WT-AIF. FIG. 8B comprises a composite black and white photograph of three representative gels run in the overlay assay, and FIG. 8C is a chart depicting the % PAR binding (relative to WT-AIF) measured for each of the mutants panel. As shown, AIF-Sm exhibited reduced binding by 40%, AIF-Dm exhibited reduced PAR binding by 80%, AIF-Tm exhibited reduced PAR binding by 90%, and AIF-Qm exhibited reduced PAR binding by 90%.

Applicants further explored PAR binding to the AIFₘ PBM by examining double amino acid mutants obtained by substituting combinations of arginine 588, lysine 589, and lysine 592 with leucine, which is more similar to the structure of arginine and lysine than it is to alanine. His-tagged WT-AIF and mutant AIF proteins were purified from E. coli and analyzed for PAR binding activity by a PAR overlay assay using purified PAR. The radioactive signal was quantified and normalized to WT-AIF, and the resulting data is represented by the chart of FIG. 8D (*** indicating p<0.001 by one-way ANOVA analysis, n=4). All double mutants (AIFR588L;K589L—reported below as SEQ. ID NO. 19, AIFR588L;K592L—reported below as SEQ. ID NO. 20, and AIFK589L;K592L—reported below as SEQ. ID NO. 21) significantly reduced PAR binding by about 85%. Similar to the triple alanine mutant (R588A; K589A; K592A—reported as SEQ. ID NO. 17), the triple leucine mutant (R588L;K589L;K592L—reported as SEQ. ID NO. 22) nearly abolished PAR-binding.

Applicants thereafter repeated the experiments using both wild type and mutant AIFₘ peptides spanning only amino acids 567-592 instead of full length AIFₘ and obtained results consistent with those using full-length AIF. Unlike the WT peptide (corresponding to only amino acids 567-592 of WT-AIF so as to consist of SEQ. ID NO. 10), the triple amino acid mutant Tm (corresponding to only amino acids 567-592 of AIF-Tm so as to consist of SEQ. ID NO. 17) failed to bind ³²P-labeled automodified PARP-1 or ³²P-labeled purified PAR. The double amino acid mutant Dm (corresponding to only amino acids 567-592 of AIF-Dm so as to consist of SEQ. ID NO. 16) bound PAR. Nitrocellulose saturation binding experiments revealed that WT peptide (Kd, 1.24×10⁻⁶M; Bmax, 4.57 pmol) and Dm peptide (Kd, 3.3×10⁻⁶M; Bmax, 3.97 pmol) bound PAR in a saturable manner, whereas the Tm peptide (Kd, 1.07×10⁻⁴M; Bmax, 1.77 pmol) had clearly reduced PAR binding. Thus, it is preferred in embodiments of the invention when conducting genetic manipulations to convert at least three target basic amino acids in the PBM to hydrophobic amino acids.

For humans, this would entail substituting target basic amino acids in the three different PBMs (represented in human AIF by the partial sequences of SEQ. ID NO. 2, SEQ. ID NO. 6, and SEQ. ID NO. 11) with hydrophic amino acids, preferably arginine or leucine. SEQ. ID NO. 22 is a partial sequence of human AIF showing the target basic amino acids for potential substitution with a hydrophic amino acid (preferably argine or leucine) as “X”, and corresponds to amino acids 223 to 245 as reported in SEQ. ID. NO. 2. SEQ. ID NO. 23 is a partial sequence of human AIF corresponding to amino acids 442 to 464 of full length human AIF showing the target basic amino acids for potential substitution in SEQ. ID. NO. 6 as “X”. Similarly, SEQ. ID NO. 24 is a partial sequence of human AIF corresponds to amino acids 568 to 590 of full length human AIF showing the target basic amino acids for potential substitution in SEQ. ID. NO. 11 as “X”. In each case, it is preferred that at least three substitutions occur, and, further, it is preferred that the substitutions are selected from either arginine or leucine.

### Example 9

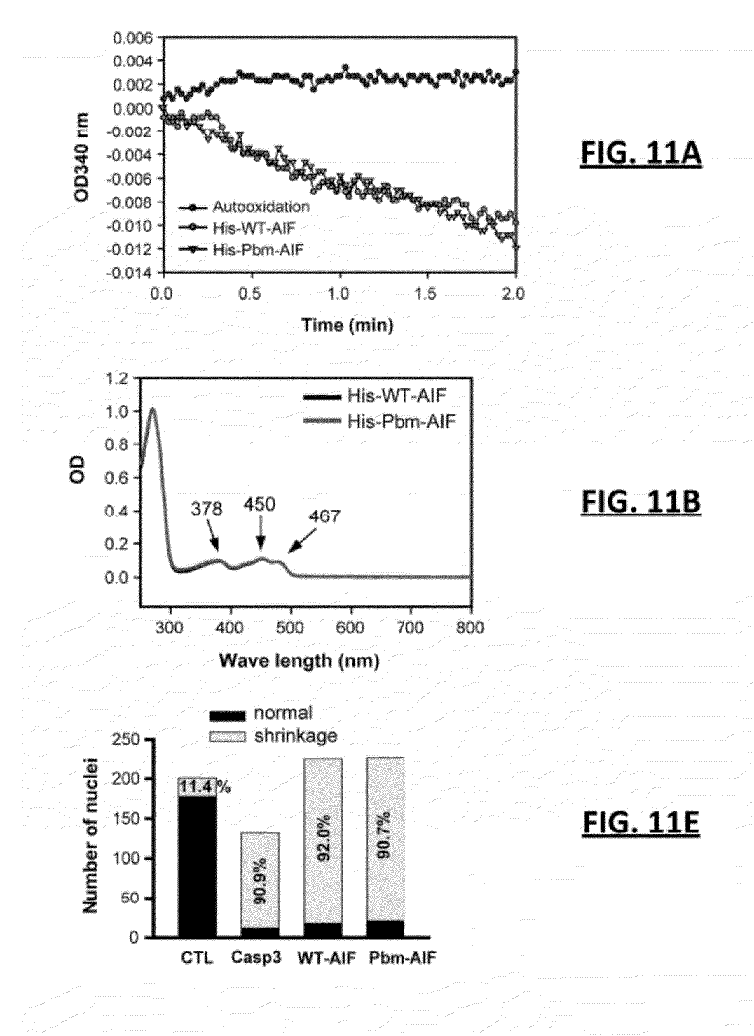

To further ascertain whether full length recombinant AIF-Tm produced above (henceforth named “Pbm-AIF”) binds PAR, Applicants performed three iterations of an EMSA assay for PAR binding with recombinant WT-AIF and Pbm-AIF, using histone H3 as a control. It was determined that WT-AIF caused a PAR mobility shift in a concentration-dependent manner, whereas Pbm-AIF had no effect on PAR mobility. FIG. 9 comprises a black and white photograph of EMSA gels showing representative results for WT-AIF and Pbm-AIF using ³²P-labeled PAR. Further, Applicants conducted a twenty percent Trisborate-EDTA (“TBE”) PAGE analysis to confirm these results, and found that WT-AIF bound to ³²P-PAR of different lengths while Pbm-AIF failed to bind to ³²P-PAR.

### Example 10

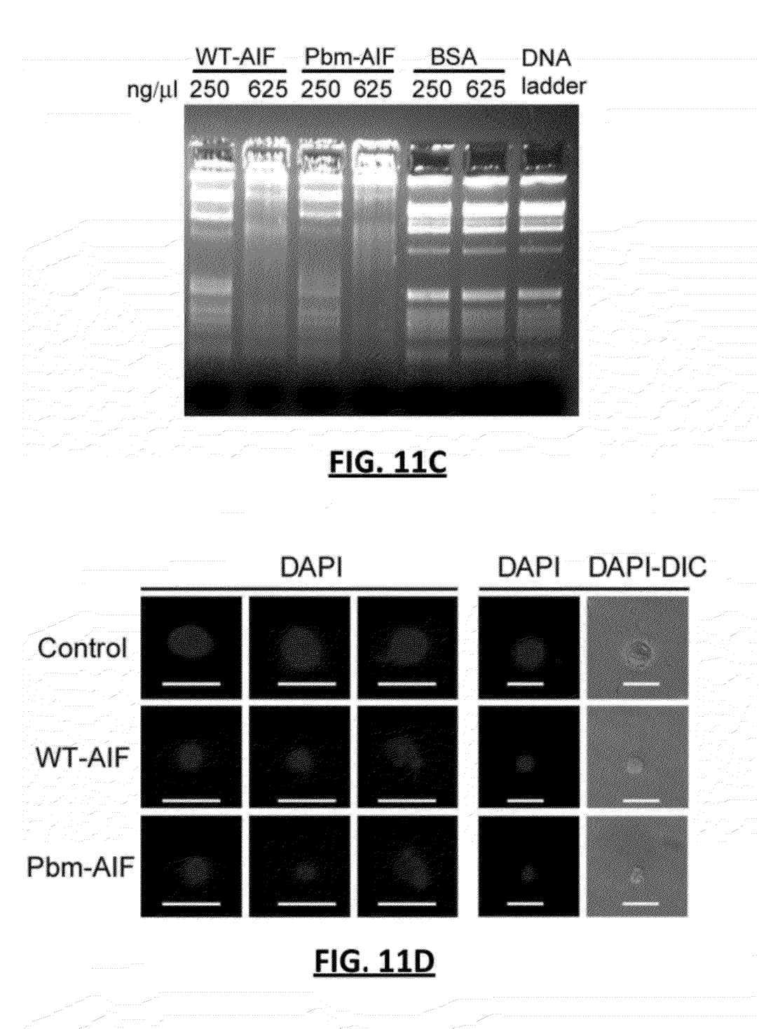

To confirm that Pbm-AIF fails to bind PAR, Applicants also performed four trials of biotin-labeled PAR pull-down assays on WT-AIF, Pbm-AIF, and histone H3. For this experiment, the pull-down assay used biotin-labeled PAR-conjugated NeutrAvidin beads. FIG. 10A comprises a composite black and white photograph depicting typical gels obtained in this pull-down assay. As can be seen in the figure, WT-AIF and histone H3 were pulled down with biotin-labeled PAR, whereas only a barely detectable amount of Pbm-AIF was pulled down. Notably, recombinant AIFs and histone H3 did not bind directly to NeutrAvidin beads in the absence of biotin-labeled PAR under the conditions used, confirming the specificity of the assay.

The PAR polymer binding properties of the mutant Pbm-AIF was then measured using full-length recombinant His-WT-AIF and His tagged Pbm-AIF (“His-Pbm-AIF”) via a series of nitrocellulose saturation binding experiments. Similar experiments were also conducted for the shorter peptides Dm and Tm in comparison to WT. FIG. 10B through FIG. 10D comprise charts reporting data from these various experiments. With regard to the shorter peptides, Applicants determined that the binding affinity constant (“Kd”) and maximum binding capacity (“Bmax”) of these AIF mimetic peptides and the results are reported in the chart of FIG. 10B. Further, as shown in FIG. 10C, Applicants discovered that full-length recombinant His-WT-AIF bound to PAR with high affinity in a saturable manner (Kd, 1.0×10⁻⁷M; Bmax, 200.83 pmol), whereas His-Pbm-AIF showed much less (and virtually linear) binding. Because the linear relation suggests that this residual PAR binding was likely non-specific, Applicants subtracted the PAR-binding values obtained with His-Pbm-AIF from those for His-WT-AIF and subjected the values thus determined for specific binding to Scatchard analysis. FIG. 10D comprises a Scatchard plot (95% confidence interval) of the results. Through this analysis it was found that the affinity of purified mouse His-WT-AIF for PAR was high, with a calculated Kd of 6.63×10⁻⁸ M and a Bmax of 116.208 pmol, a concentration consistent with those found in intact HeLa cells after MNNG treatment or in cortical neurons after exposure to NMDA.

### Example 11

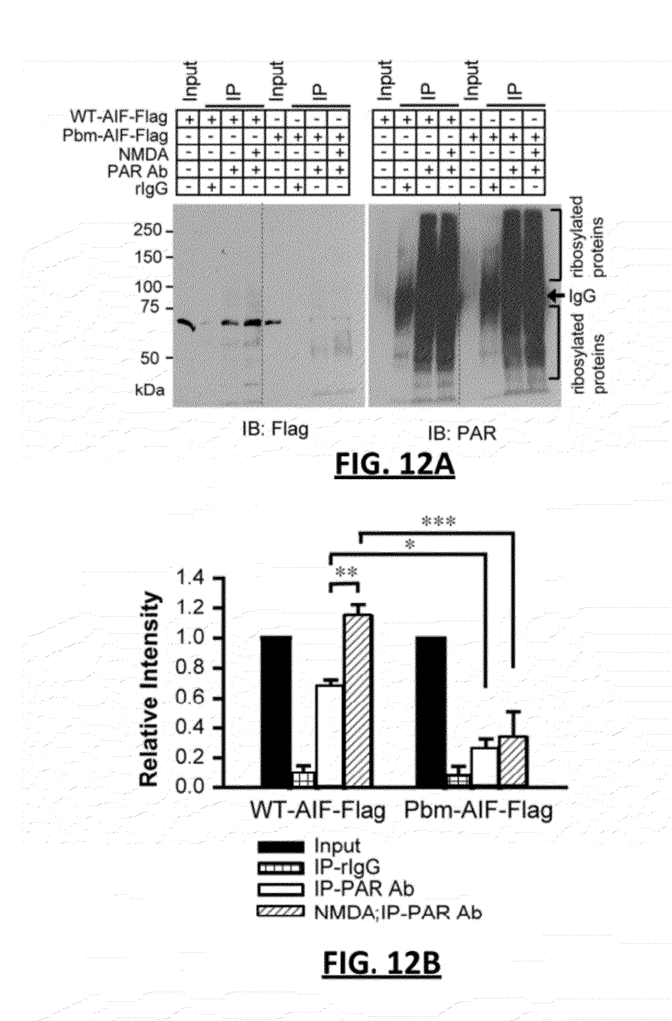

Independent of its role in parthanatos, AIF has NADH (the reduced form of nicotinamide adenine dinucleotide) oxidase activity, binds to FAD (flavin adenine dinucleotide) and DNA, and causes nuclear condensation. To determine whether the triple amino acid mutation found in Pbm-AIF interferes with these properties, Applicants assessed the NADH oxidase activity, FAD-binding capacity, and DNA-binding capacity of full-length His-WT-AIF and His-Pbm-AIF. First, the NADH oxidase activity of His-WT-AIF and His-Pbm-AIF was visualized on native gel by NBT reduction, using BSA as a negative control. Compared to His-WT-AIF, His-Pbm-AIF showed no significant change in NADH oxidase activity, as reported in the color graph of experiment results which is FIG. 11A (NADH oxidase activity reported therein being determined by monitoring the changes in absorbance at OD 340 nm).

FAD binding of His-WT-AIF and His-Pbm-AIF was determined by spectrophotometric wave length scanning of purified proteins. Again, as shown in the color diagram of FIG. 11B, there was no significant difference identified between the FAD binding properties of His-WT-AIF and His-Pbm-AIF. As shown, the red curve for His-Pbm-AIF almost perfectly matches, and thus largely obscures in the diagram, the blue curve for His-WT-AIF.

Further, a DNA retardation assay of His-WT-AIF and His-Pbm-AIF was conducted by incubating both with DNA (200 ng) for 30 minutes, using BSA as a negative control. FIG. 11C comprises a black and white photograph of a representative gel for this DNA retardation assay. Again, as shown by the gel, the DNA-binding properties of both His-WT-AIF and His-Pbm-AIF were found to be comparable.

Finally, non-tagged WT-AIF and Pbm-AIF were both found to cause comparable levels of chromatin condensation and nuclear shrinkage when incubated with isolated HeLa nuclei. FIG. 11D comprises an array of color photographs showing the result of fluorescence microscopy for various cultures, as labeled in the figure, following DAPI staining. As can be seen, the blue areas for each of the WT-AIF and Pbm-AIF images are of comparable size and intensity (scale bar therein indicates 20 μm). FIG. 11E is a chart reporting the result of a quantification of nuclei treated by WT-AIF, Pbm-AIF, and control (“CTL”), using Caspase 3 as a positive control.

Importantly, these results together indicate that AIF is a high affinity PAR binding protein and that the triple amino acid (R588A;K589A;K592A) mutation of AIFₘ termed herein Pbm-AIF substantially eliminates PAR binding without affecting the PAR-independent functions of AIFₘ.

### Example 12

To determine whether Pbm-AIF fails to bind to PAR in cultured cortical neurons, neuronal cultures were transduced by lentivirus with WT-AIF-Flag or Flag-tagged Pbm-AIF (“Pbm-AIF-Flag”). Neurons from Hq mice, which have an 80% reduction in WT-AIF due to a proviral insertion, were used for these experiments to reduce interference from endogenous AIF. Green fluorescent protein (“GFP”) lentivirus-transduced and non-transduced Hq neurons served as negative controls. AIF was translocation to the nucleus for each neuron culture was monitored 2 hours after administration of NMDA (500 μM NMDA for 5 minutes). Applicants found that WT-AIF-Flag and Pbm-AIF-Flag were equally abundant in Hq cortical neurons and both localized to the mitochondria, as determined by their co-localization with the mitochondrial enzyme manganese superoxide dismutase (“MnSOD”).

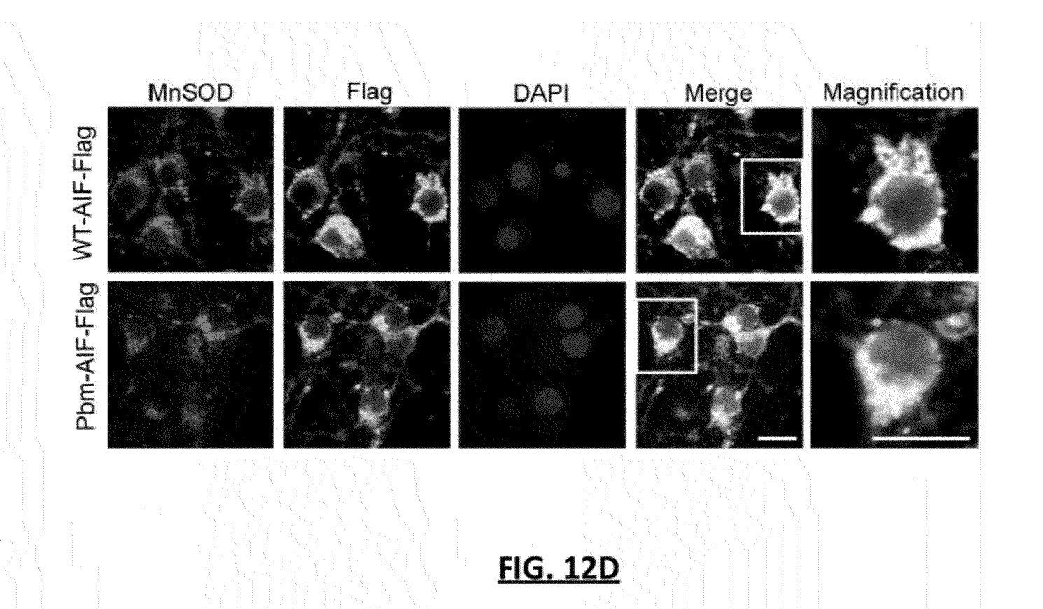

Specifically, treatment of Hq cortical cultures with NMDA activated PARP-1, leading to the time-dependent formation of PAR in both the nuclear fraction and the postnuclear fraction. Two hours after a 5 minute treatment with 500 μM NMDA, Applicants measured PAR binding to AIF from postnuclear fractions and compared it to that in control (non-NMDA-treated) cultures via co-immunoprecipitation with rabbit IgG, and FIG. 12A is a black and white gel photograph of representative gels for this test. The signal from five such assays was quantified, and the results of which (normalized to input) are presented in the graph of FIG. 12B. As can be seen, immunoprecipitation with PAR pulled down WT-AIF-Flag from both non-NMDA- and NMDA-treated cultures, whereas Pbm-AIF-Flag failed to appreciably co-immunoprecipitate with PAR. Further, WT-AIF-Flag interacted with PAR in un-stimulated Hq cortical neurons, and this interaction significantly increased following NMDA treatment.

To confirm that the PAR-AIF interaction was not mediated by DNA- or RNA-binding, Applicants performed a co-immunoprecipitation of PAR with WT-AIF-Flag for the cortical neurons. For this assay, post-nuclear proteins were isolated 2 hours after NMDA treatment (again, 500 μM NMDA 5 minutes) and precipitated by anti-PAR antibody in the presence of or absence of BDRE cocktail containing 1 mg/ml benzonase, 4 U/ml DNAse I, 10 ml/ml RNAse A, and 0.5 μg/ml ethidium bromide. The immunoblot was probed with an anti-Flag antibody and re-probed with the anti-PAR antibody. FIG. 12C comprises a black and white photograph of a representative gel for this assay. As can be seen, the third and fourth columns of the gel are virtually unchanged, indicating no significant effect of the BDRE cocktail, which Applicants accepted as an indication that the observed interaction was not mediated by DNA- or RNA-binding.

Specifically, the mitochondrial location of exogenous WT-AIF-Flag and Pbm-AIF-Flag in Hq primary cortical neuronal culture was determined by immunostaining the cells with mitochondrial protein MnSOD (pink) and AIF-Flag (yellow). Nuclei were counterstained with DAPI (blue). FIG. 12D comprises an array of 10 color fluorescence photographs that are representative for at least 4 independent experiments. In those photographs (scale bar indicates 10 μm), pink indicates MnSOD, yellow indicates AIF-Flag, and white indicates overlap between MnSOD and AIF staining. Analysis of the images found that both endogenous WT-AIF and exogenous Pbm-AIF translocated to the nucleus 24 hours after 100 nM staurosporine treatment.

Applicants obtained similar results from the treatment of HeLa cells transduced with WT-AIF-Flag or Pbm-AIF-Flag with MNNG. PAR co-immunoprecipitated with WT-AIF-Flag but not with Pbm-AIF-Flag in postnuclear fractions of both non-MNNG- and MNNG-treated cultures, and quantification revealed that this interaction was significantly increased by MNNG treatment. Rabbit IgG, used as a negative control in both Hq cortical cultures and HeLa cells, failed to co-immunoprecipitate with WT-AIF-Flag or Pbm-AIF-Flag. Together, these results indicate that Pbm-AIF fails to bind to PAR either in cells at rest or following PARP-1 activation.

### Example 13

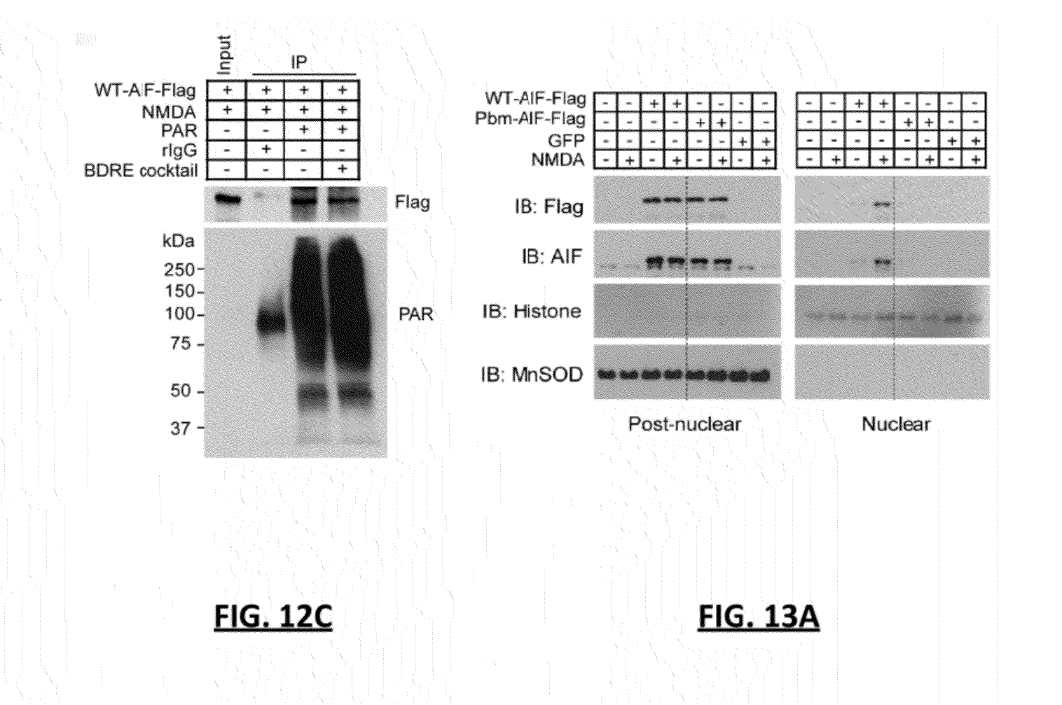

Applicants next determined whether Pbm-AIF was released from the mitochondria and translocated to the nucleus after PARP-1 activation in Hq mouse cortical neuronal cultures transduced with WT-AIF-Flag or Pbm-AIF-Flag lentiviruses. GFP lentivirus-transduced and non-transduced neurons served as negative controls. Again, AIF translocation to the nucleus was monitored two hours after NMDA (500 μM, 5 minutes) treatment sufficient to initiate parthanatos in wild-type cells. As shown in the gel of FIG. 13A, immunoblot analysis of nuclear and postnuclear fractions using antibodies to Flag or AIF revealed that WT-AIF-Flag translocated to the nucleus following NMDA administration, whereas Pbm-AIF-Flag failed to do so.

Moreover, confocal image analysis confirmed that, two hours after NMDA stimulation (500 μM, 5 minutes), WT-AIF-Flag was observed in the nucleus whereas Pbm-AIF-Flag remained in the cytosol. FIG. 13B comprises an array of 16 color fluorescence photographs for the indicated cells, using DAPI (blue) nuclei staining and Flag being shown in red (scale bars indicate 20 μm). As a control, Applicants reran the above analysis for both WT-AIF-Flag and Pbm-AIF-Flag using 100 nM staurosporine instead of NMDA stimulation. The staurosporine caused both forms of AIF to translocate from the mitochondria to the nucleus 24 hours after treatment. This indicated that both forms of AIF are able to translocate, but in parthanatos PAR binding to AIF is required for such translocation.

Finally, endogenous AIF, WT-AIF-Flag, and Pbm-AIF-Flag were monitored for nuclear translocation by confocal image analysis, following MNNG treatment (50 μM for 15 minutes) of HeLa cells under conditions in which MNNG induces parthanatos. In cells transfected with WT-AIF-Flag, both endogenous AIF and WT-AIF-Flag translocated to the nucleus 4 hours following MNNG treatment. Further, the PARP-1 inhibitor, 3,4-Dihydro-5[4-(1-piperindinyl)butoxy]-1(2H)-isoquinoline, (“DPQ”), prevented the nuclear translocation of both endogenous AIF and WT-AIF-Flag, confirming the dependence of AIF translocation on PARP-1 activation. In contrast, MNNG treatment failed to induce the nuclear translocation of Pbm-AIF-Flag, despite inducing the translocation of endogenous AIF. This indicates that Applicant's identified PBM of AIF is responsible for parthanatos-associated translocation of AIF.

### Example 14

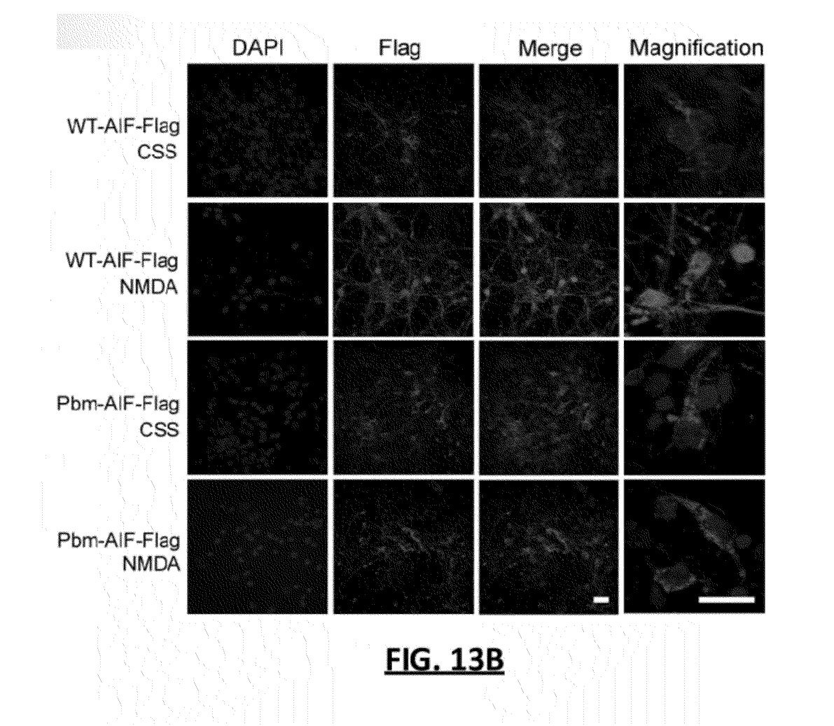

Next, Applicants used Hq cortical neurons transduced with WT-AIF-Flag or Pbm-AIF-Flag lentiviruses to determine whether PAR plays a direct role in mitochondrial AIF release. For this experiment, mitochondria isolated from cortical neurons, and GFP lentivirus transduced and non-transduced neurons served as negative controls. Three days after lentivirus infection, mitochondria were isolated and exposed to purified PAR to elicit in vitro AIF release by incubation with PAR for 30 minutes, and the levels of AIF were determined in the mitochondria and in the supernatant. FIG. 14 comprises a black and white photograph of a representative gel obtained in one of the 6 independent experiments run. It shows that WT-AIF-Flag and Pbm-AIF-Flag were equally abundant in mitochondria (labeled “Mit” in FIG. 14A). However, PAR induced the release of WT-AIF-Flag from mitochondria but failed to release Pbm-AIF-Flag in the supernatant (labeled “SN” in FIG. 14). Similar results were observed in HeLa cells transiently transfected with WT-AIF-Flag or Pbm-AIF-Flag. Two days after transfection, mitochondria were isolated and exposed to purified PAR, which induced the release of WT-AIF-Flag, but failed to release Pbm-AIF-Flag. Together, these results indicate that PAR binding to AIF is required for AIF release following PARP-1 activation.

### Example 15

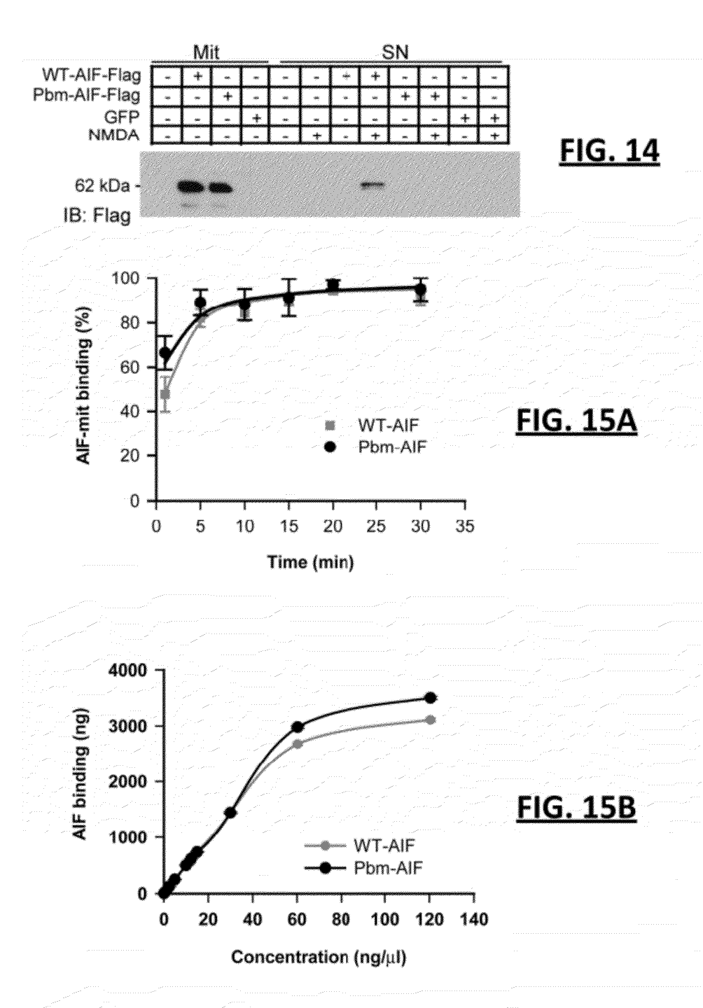

One commonly accepted estimate is that approximately 20%-30% of AIF resides on the cytosolic side of the outer membrane of mitochondria, where it is poised to be rapidly released by PAR following PARP-1 activation. Applicants thus wanted to perform tests of AIF binding to mitochondria determine how readily purified WT-AIF and Pbm-AIF bind to mitochondria in a time- and concentration-dependent manner. Specifically, recombinant WT-AIF (2.5 ng/μl) and Pbm-AIF (2.5 ng/μl) were incubated with isolated mitochondria (200 μg/ml) for 1 minute, 5 minutes, 10 minutes, 15 minutes, 20 minutes or 30 minutes. The levels of AIF were determined in the supernatant after each interval, and the amount of respective AIF binding to the mitochondria was calculated therefrom. The graph of FIG. 15A provides data obtained from 3 independent trials, and shows that after 5 minutes the binding percentage of WT-AIF and Pbm-AIF were equivalent. Similarly, isolated mitochondria (200 μg/ml) were incubated with different concentrations of recombinant WT-AIF and Pbm-AIF varying from 0.1 ng/μl to 120.5 ng/μl at room temperature for 30 minutes, and the levels of AIF were determined in the supernatant. The results of this portion of the experiment is provided by the graph of FIG. 15B. As shown, both forms of AIF bound to the mitochondria in a concentration dependent manner.

### Example 16

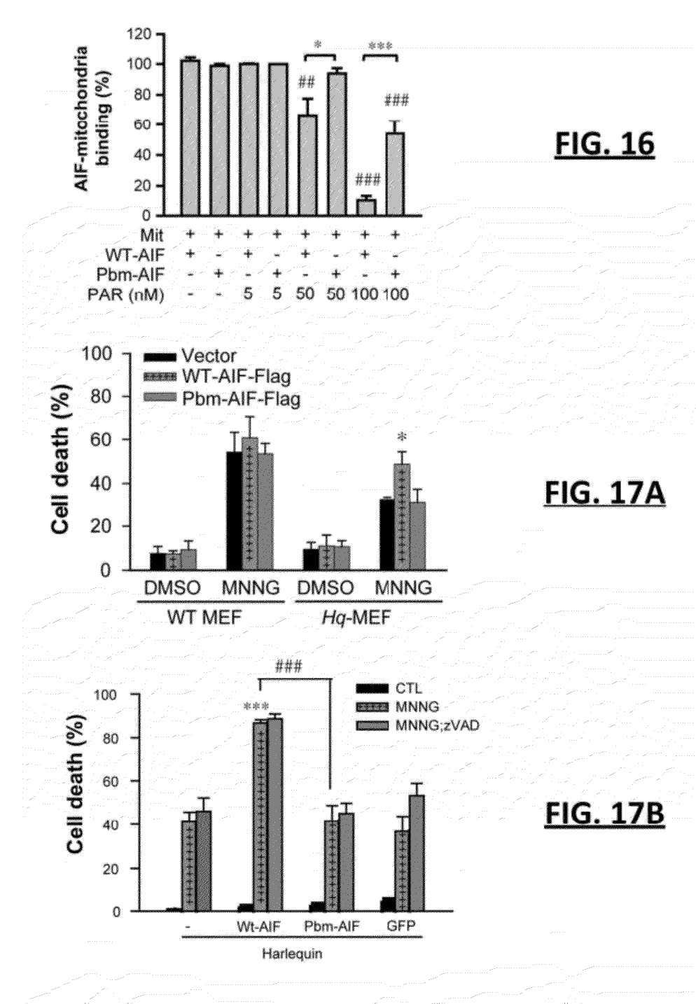

To investigate the mechanism underlying mitochondrial AIF release following PARP-1 activation, Applicants incubated isolated mitochondria with purified non-tagged forms of AIF in the presence or absence of PAR and followed this by centrifugation to remove the mitochondria. Detection of AIF remaining in the supernatant was thereafter performed through immunoblot analysis. The results of this experiment is depicted in the graph of FIG. 16, which reports that untreated mitochondria effectively depleted both WT-AIF and Pbm-AIF from the supernatant (see the first two columns on the left), again confirming that both WT-AIF and Pbm-AIF bind to mitochondria. With regard to mitochondria treated with PAR, it was found that 5 nM PAR failed to affect association of WT-AIF or Pbm-AIF with mitochondria, whereas 50 nM PAR increased the concentration of WT-AIF in the supernatant, indicating that it disrupted its binding to mitochondria, with barely detectable effects on the concentration of Pbm-AIF (see the fifth and sixth columns from the left of FIG. 16. Further, 100 nM PAR almost completely abolished the ability of WT-AIF to bind to mitochondria and partially reduced mitochondrial binding of Pbm-AIF (see the last two columns).

The supernatant collected in the experiments of Example 16 were also analyzed for the presence of Tom20 and cyclochrome c. It was found that 5 nM and 50 nM PAR failed to induce the release of endogenous cytochrome c from mitochondria under the same experimental conditions, although a weak cytochrome c signal could be detected with exposure to 100 nM PAR. Mitochondrial outer membrane protein Tom20 was also used to monitor mitochondrial integrity and it could not be detected in the supernatant at any concentration of PAR used, indicating that mitochondrial integrity was maintained throughout the experiment. These data collectively suggest that PAR can disrupt binding of WT-AIF to intact mitochondria, thereby causing AIF release, whereas Pbm-AIF is relatively resistant to the releasing effects of PAR.

### Example 17

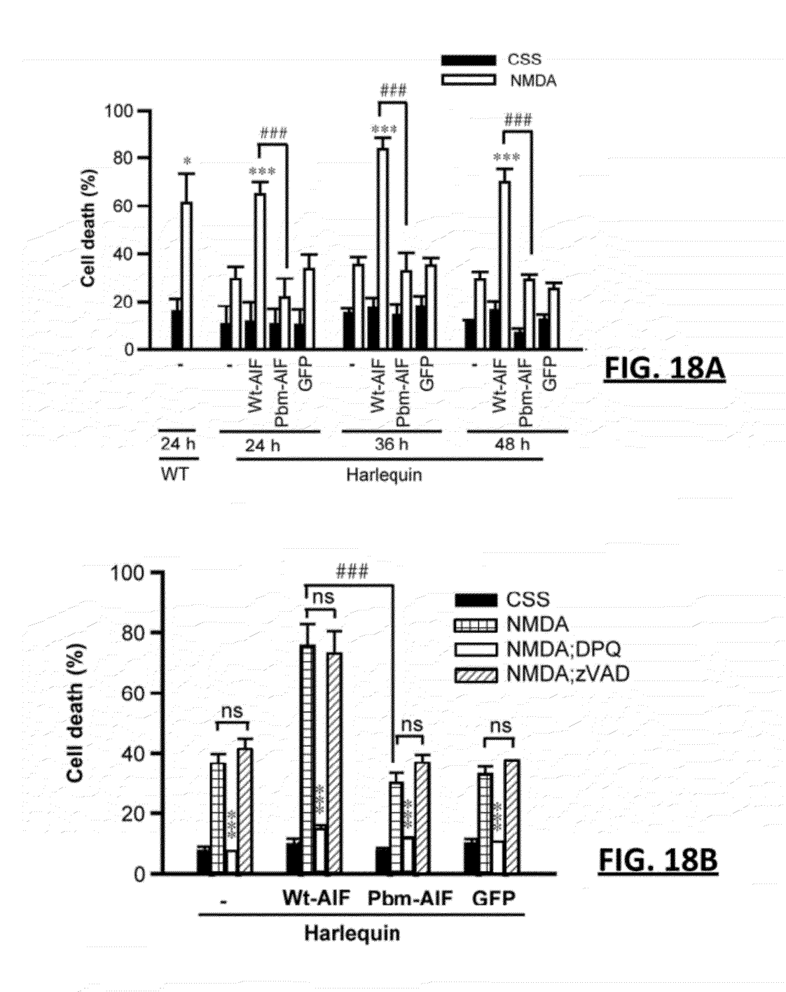

To ascertain whether the failure of the Pbm-AIF to translocate to the nucleus following PARP-1 activation has implications for PARP-1-dependent cell death, Applicants monitored the susceptibility of cells expressing WT-AIF-Flag or Pbm-AIF-Flag to parthanatos. Using WT mouse embryonic fibroblasts (“MEFs”), 24 hours after MNNG cytotoxicity (500 μM for 15 min) WT-AIF-Flag and Pbm-AIF-Flag were equally abundant yet failed to affect susceptibility of WT MEFs to MNNG-induced cell death, as shown in the graph of FIG. 17A. Hq MEFs showed significantly reduced cell death in response to MNNG compared to WT MEFs, however WT-AIF-Flag restored their susceptibility to MNNG toxicity. In contrast, Pbm-AIF-Flag had no discernible effect on death of Hq MEFs. Additionally, WT-AIF-Flag and Pbm-AIF-Flag were found to be equally expressed in both HQ and WT MEF cells.

Further, the effect WT-AIF-Flag or Pbm-AIF-Flag on Hq MEF cell viability was determined 24 hours after MNNG (500 μM for 15 min) cytotoxicity with and without zVAD (100 μM), and the results of three trials is reported in FIG. 17B. As shown in FIG. 17B, the broad-spectrum caspase inhibitor zVAD also failed to influence MNNG-induced cell death in MEFs, further confirming a lack of involvement of caspases in parthanatos.

### Example 18

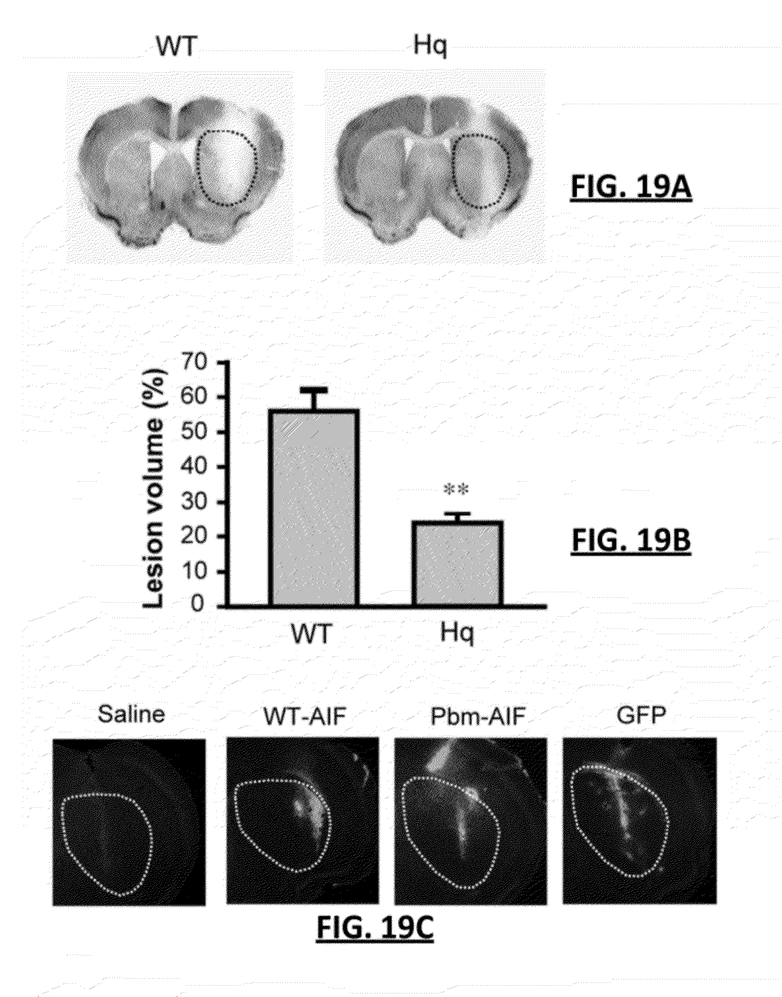

Applicants next assessed NMDA excitotoxicity in Hq cortical neurons, which are known to be resistant to NMDA excitotoxicity, transduced with WT-AIF-Flag or Pbm-AIF-Flag. HQ cortical neuron cells were transduced with lentivirus carrying WT-AIF-Flag or Pbm-AIF-Flag. Non-transduced neurons and GFP-lentivirus infected cells were used as negative controls. FIG. 18A comprises an array of eight representative color fluorescence photographs showing the effect of WT-AIF-Flag or Pbm-AIF-Flag on NMDA-induced cytotoxicity in Hq cortical neurons 24 hours after NMDA (500 μM for 5 min). In those photos, blue indicates Hoechst 33342 staining and red indicates propidium iodide staining. The neurons transduced with WT-AIF show significantly more red (proportional to the amount of parthanatos cells death). The cytotoxicity in these Hq cortical neurons was measured at 24 hours, 36 hours, and 48 hours after NMDA (500 μM for 5 min) treatment, and the data for this is reported in the graph of FIG. 18B. It can be seen therein that Pbm-AIF had no appreciable affect (“ns” designates not significant) on the percentage of cell death following treatment with NMDA while WT-AIF increased sell death significantly (“###” designates p<0.001 by one-way ANOVA). These experiments show that WT-AIF-Flag restored Hq cortical neuron susceptibility to NMDA excitotoxicity, whereas Pbm-AIF-Flag failed to do so.

Additionally, Applicants repeated the assays including DPQ (30 μM) or zVAD (100 μM) in additional to NMDA. As expected, DPQ, a PARP-1 inhibitor, inhibited NMDA-induced cortical neuron death, but zVAD did not (*** indicates p<0.001) as compared to its control group treated with CSS.

### Example 19

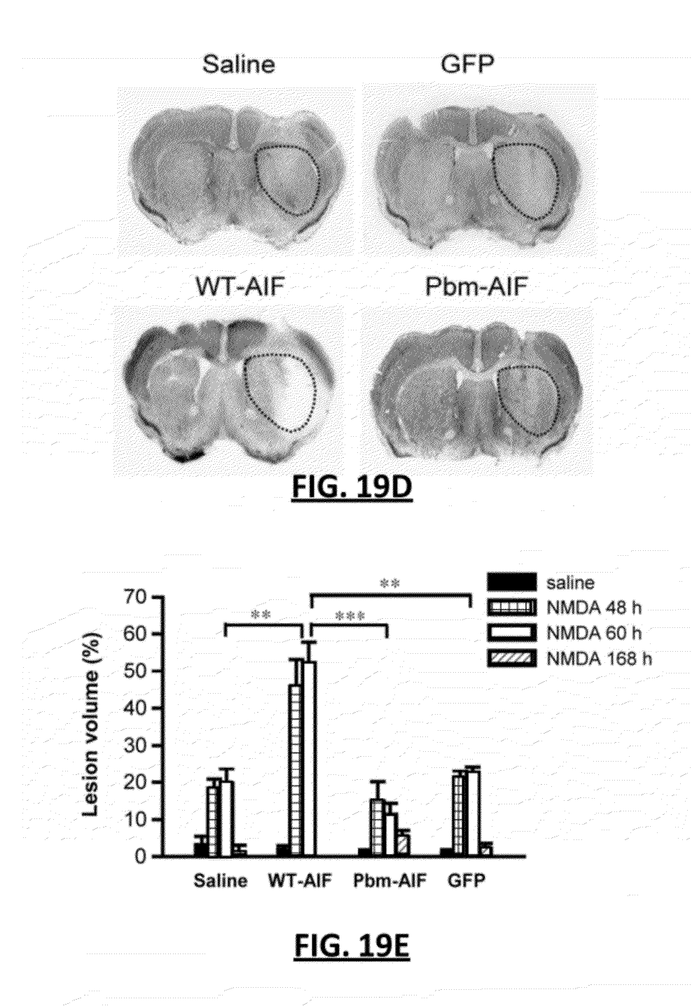

Applicants used Hq mice to determine whether the in vitro findings described above are relevant in vivo. Specifically, Applicants designed experiments to investigate NMDA receptor-mediated glutamate excitotoxicity, which is a major component of neuronal death in neurodegenerative diseases and stroke. For this experiment, NMDA (20 nmol) was administered stereotactically into the striata of Hq or WT mice. 60 hours later, Applicants assessed lesion volume in the striatum by Nissl staining. FIG. 19A comprises representative color photographs of the lesions in both types of mice after Nissl staining (with the dotted line outlining the striatum), and FIG. 19B reports the relative average lesion volumes obtained experimentally in both mice. Notably, NMDA administration caused a larger lesion in WT mice than in Hq mice.

Applicants next assessed whether replacement of WT-AIF-Flag or Pbm-AIF-Flag through lentiviral transduction would restore the sensitivity of Hq mice to NMDA excitotoxicity. Lentiviruses carrying WT-AIF-Flag, Pbm-AIF-Flag, or GFP were injected in both the left and right side striata of Hq mice seven days before injection with NMDA in the right striatum and saline in the left striatum. Before exposing the transduced mice to NMDA, Applicants confirmed via imaging that WT-AIF-Flag, Pbm-AIF-Flag, and GFP were successfully expressed in striatum, as shown in the color photographs of FIG. 19C, with the dotted line outlining the striatum.

Applicants found that WT-AIF-Flag restored sensitivity to NMDA excitotoxicity, so that Hq mice showed larger lesions volume 48 hours or 60 hours after the NMDA injection. FIG. 19D representative color photographs, with Nissl staining, of the striatum 60 hours after NMDA injection (20 nmol), with the dotted line outlining the striatum. FIG. 19E in turn summarizes the data regarding lesion volumes obtained from WT-AIF-, Pbm-AIF-, GFP-, and saline-injected mice assessed at 48 hours, 60 hours, and 168 hours after injection of NMDA or saline (11 mice given WT-AIF, 10 mice given Pbm-AIF, 9 mice given saline, and 9 mice given GFP). While WT-AIF-Flag restored sensitivity to NMDA, Pbm-AIF-Flag failed to restore NMDA excitotoxicity. Further, lesions following NMDA injection were similar to those seen in mice transduced with GFP or injected with saline. Mice injected with Pbm-AIF-Flag lentivirus survived for at least 168 hours after the NMDA injection, whereas, with one exception, mice injected with WT-AIF-Flag lentivirus failed to survive for 168 hours. Also, the lesion volume of mice injected with Pbm-AIF-Flag lentivirus remains reduced at 168 hours, suggesting that the protection is long-lasting.

### Example 20

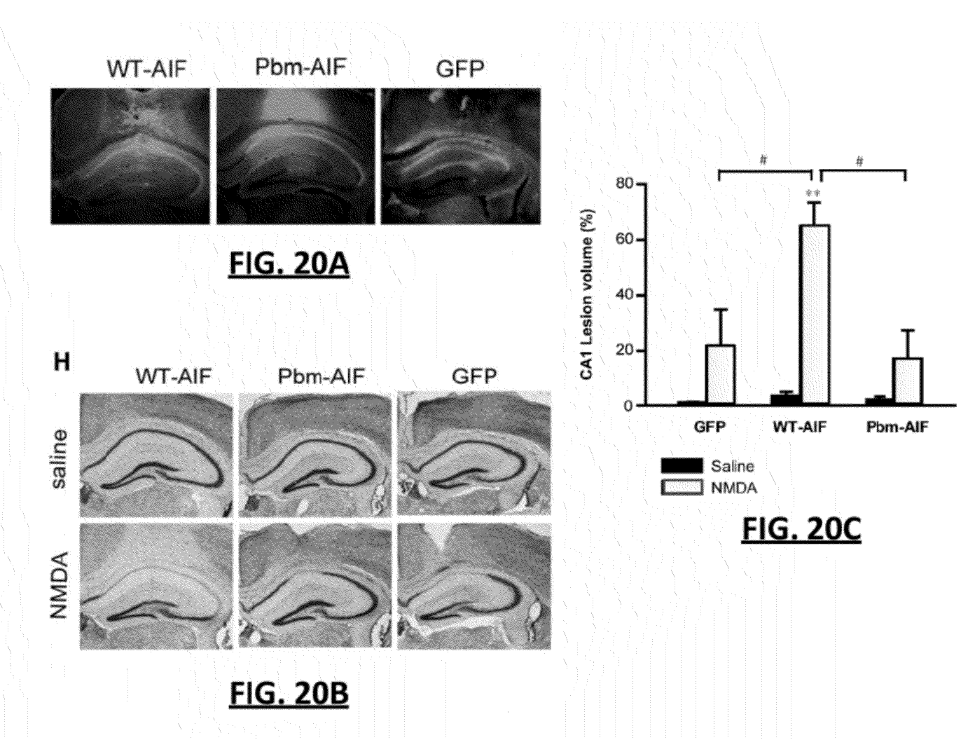

In a second model of NMDA excitotoxicity, Applicants injected adeno-associated virus (“AAV2”) carrying WT-AIF-Flag, Pbm-AIF-Flag, or GFP into the CA1 region of both the left and right hippocampus of Hq mice. WT-AIF-Flag, Pbm-AIF-Flag, and GFP were strongly transduced throughout the whole CA1 region, as shown by the color photos depicted in FIG. 20A. Seven days following virus injection, Applicants injected NMDA into the right CA1 region and saline into the left CA1 region, and then assessed CA1 lesion volume 60 hours later. NMDA injection caused a large lesion volume in WT-AIF-Flag injected Hq mice, whereas Pbm-AIF-Flag- and GFP-injected Hq mice had significantly smaller and equivalent lesions. FIG. 20B is an array of six representative Nissl staining photos taken 60 hours post NMDA injection, while FIG. 20C is a graph of the experimental data obtained for lesion volume percentages. Together, the results indicate that PAR binding to AIF is required for NMDA excitotoxicity both in vitro and in vivo.

The various experiments having thus been described above, one of ordinary skill in the art will readily appreciate that Applicants have established for the first time a link between parthanatos and the binding of PAR polymer to AIF found on the outer mitochondrial surface. Applicants have also established that inhibiting this binding, such as through genetic manipulation of the sequence of AIF in target cells or alternatively through administration of drug products that inhibit such binding, can inhibit parthanatos without interfering with the other beneficial cell functions of PAR or AIF. Further, Applicants have established that AIF release from the mitochondrial surface into cytosol causes the release AIF to translocate to the cell nucleus and cause cell death. Therefore, one skilled in the art will likewise appreciate that genetic modifications to AIF or drug therapies which cause AIF to bind less readily to the outer mitochondrial membrane or to otherwise make AIF more readily release from the mitochondrial membrane could be applied to cause the death of target cells, such as in anti-tumor or anti-cancer treatments.

Further, one of ordinary skill in the art will readily appreciate that Applicants' discoveries regarding the effect and nature of PAR binding to AIF in relation to parthanatos can be used to identify small molecules that mimic PAR to induce the release of AIF. These PAR-mimics could be used as therapeutic active ingredients to therapeutically induce cell death in a target group of cells, such as in cancerous and other tumors, including solid tumors, advanced solid tumors, hepatocellular carcinoma, prostate cancer, colorectal cancer, ovarian cancer, breast cancer, BRCA-1 or -2 associated breast cancer, triple negative breast cancer, skin cancer, metastatic melanoma, advanced solid tumors, non-hematologic malignancies, brain neoplasms, pancreatic advanced tumors, pancreatic neoplasms, gastric cancer, melanoma neoplasms, breast neoplasms, ovarian neoplasm, neoplasm metastasis, glioblastoma multiforme, lymphoma, and squamous cell lung cancer.

Further, one of ordinary skill in the art will readily appreciate that Applicants' discoveries regarding the effect and nature of PAR binding to AIF in relation to parthanatos can be used to identify small molecules, biologics, and other therapeutics that inhibit the binding of PAR with AIF and thus prevent PAR from inducing the release of AIF. These PAR-AIF binding inhibitors could be used as therapeutic active ingredients to treat the following diseases and conditions: diabetes and conditions relating thereto, including diabetes mellitus, diabetic retinopathy, diabetic endovascular disease, diabetic nephropathy, and diabetic neuropathy; and cardiovascular or cardiac diseases and conditions including acute myocardial infarction, heart disease, cardiac allograft rejection, postoperative cardiac complications (such as in conjunction with cardiac bypass surgery), myocarditis, heart failure, atherosclerosis, vascular hyporeactivity in sepsis, and circulatory shock.

Additionally, such PAR-AIF binding inhibitors could be used as therapeutic active ingredients to treat the following diseases and conditions of the central nervous system (“CNS”): stroke, ischemia reperfusion injury (e.g., of the brain, spinal cord, retina, muscle, kidney, or heart), postoperative CNS complications, traumatic brain injury, spinal cord injury, Parkinson's disease, Alzheimer's disease, multiple sclerosis, retinopathy, and macular degeneration. Finally, such PAR-AIF binding inhibitors could be used as therapeutic active ingredients to treat the following neurodegenerative and related neurologic diseases: Alexander's disease, Alper's disease, Alzheimer's disease, amyotrophic lateral sclerosis, ataxia telangiectasia, Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Sandhoff's disease, Schilder's disease, subacute combined degeneration of spinal cord secondary to pernicious anaemia, schizophrenia, spinocerebellar ataxia, spinal muscular atrophy, Steele-Richardson-Olszewski disease, and tabes dorsalis. Other diseases associated with parthanatos, including, arthritis and colitis would also be treatable by administration of effective amounts of PAR-AIF inhibitors.

Embodiments of the invention thus can include methods for treating any of the above diseases and/or conditions by administering an effective amount of a PAR-AIF binding inhibitor to a patient having one of the above diseases for the purpose of treating or alleviating parthanatos-related symptoms. Additionally, embodiments of the invention can include methods for treating any of the above diseases and/or conditions by genetically modifying the PBM of AIF present in target cells of a patient to prevent or reduce the binding of PAR to such modified AIF in those target cells for the purpose of treating or alleviating parthanatos-related symptoms. Further, embodiments of the invention can include methods for identifying agents effective to treat parthanatos-related symptoms associated with any of the above diseases and/or conditions by testing prospective PAR-AIF binding inhibitors for their ability to bind to the PBM of AIF, such as by testing the binding of prospective inhibitors to peptides comprising the amino acid sequences of SEQ. ID NO. 2, SEQ. ID NO. 6, and/or SEQ. ID NO. 11.

Having described preferred embodiments of the invention, it will now become apparent to those of ordinary skill in the art that other embodiments incorporating these concepts may be used. Accordingly, it is submitted that that the invention should not be limited to the described embodiments but rather should be limited only by the spirit and scope of the appended claims.

Thus, although the invention has been described and illustrated with a certain degree of particularity, it is understood that the present disclosure has been made only by way of example, and that numerous changes in the combination and arrangement of steps, ingredients, or processes can be resorted to by those skilled in the art without departing from the spirit and scope of the invention, as will be claimed hereafter.

### REFERENCES

K. K. David, S. A. Andrabi, T. M. Dawson, V. L. Dawson, Parthanatos, a messenger of death. Front Biosci 14, 1116-1128 (2009).
Y. Wang, V. L. Dawson, T. M. Dawson, Poly(ADP-ribose) signals to mitochondrial AIF: a key event in parthanatos. Exp Neurol 218, 193-202 (2009).
P. Pacher, C. Szabo, Role of the peroxynitrite-poly(ADP-ribose) polymerase pathway in human disease. Am J Pathol 173, 2-13 (2008).
E. C. Cheung, N. Joza, N. A. Steenaart, K. A. McClellan, M. Neuspiel, S. McNamara, J. G. MacLaurin, P. Rippstein, D. S. Park, G. C. Shore, H. M. McBride, J. M. Penninger, R. S. Slack, Dissociating the dual roles of apoptosis-inducing factor in maintaining mitochondrial structure and apoptosis. Embo J 25, 4061-4073 (2006).
Y. H. Kang, M. J. Yi, M. J. Kim, M. T. Park, S. Bae, C. M. Kang, C. K. Cho, I. C. Park, M. J. Park, C. H. Rhee, S. I. Hong, H. Y. Chung, Y. S. Lee, S. J. Lee, Caspase-independent cell death by arsenic trioxide in human cervical cancer cells: reactive oxygen species-mediated poly(ADP-ribose) polymerase-1 activation signals apoptosis-inducing factor release from mitochondria. Cancer Res 64, 8960-8967 (2004).
S. W. Yu, H. Wang, M. F. Poitras, C. Coombs, W. J. Bowers, H. J. Federoff, G. G. Poirier, T. M. Dawson, V. L. Dawson, Mediation of poly(ADP-ribose) polymerase-1-dependent cell death by apoptosis-inducing factor. Science 297, 259-263 (2002).
D. Brown, B. D. Yu, N. Joza, P. Benit, J. Meneses, M. Firpo, P. Rustin, J. M. Penninger, G. R. Martin, Loss of AIF function causes cell death in the mouse embryo, but the temporal progression of patterning is normal. Proc Natl Acad Sci USA 103, 9918-9923 (2006).
S. A. Andrabi, N. S. Kim, S. W. Yu, H. Wang, D. W. Koh, M. Sasaki, J. A. Klaus, T. Otsuka, Z. Zhang, R. C. Koehler, P. D. Hurn, G. G. Poirier, V. L. Dawson, T. M. Dawson, Poly(ADP-ribose) (PAR) polymer is a death signal. Proc Natl Acad Sci USA 103, 18308-18313 (2006).
E. C. Cheung, L. Melanson-Drapeau, S. P. Cregan, J. L. Vanderluit, K. L. Ferguson, W. C. McIntosh, D. S. Park, S. A. Bennett, R. S. Slack, Apoptosis-inducing factor is a key factor in neuronal cell death propagated by BAX-dependent and BAX-independent mechanisms. J Neurosci 25, 1324-1334 (2005).
C. Culmsee, C. Zhu, S. Landshamer, B. Becattini, E. Wagner, M. Pellecchia, K. Blomgren, N. Plesnila, Apoptosis-inducing factor triggered by poly(ADP-ribose) polymerase and Bid mediates neuronal cell death after oxygen-glucose deprivation and focal cerebral ischemia. J Neurosci 25, 10262-10272 (2005).
G. Cao, J. Xing, X. Xiao, A. K. Liou, Y. Gao, X. M. Yin, R. S. Clark, S. H. Graham, J. Chen, Critical role of calpain I in mitochondrial release of apoptosis-inducing factor in ischemic neuronal injury. J Neurosci 27, 9278-9293 (2007).
A. Joshi, V. Bondada, J. W. Geddes, Mitochondrial micro-calpain is not involved in the processing of apoptosis-inducing factor. Exp Neurol 218, 221-227 (2009).
Y. Wang, N. S. Kim, X. Li, P. A. Greer, R. C. Koehler, V. L. Dawson, T. M. Dawson, Calpain activation is not required for AIF translocation in PARP-1-dependent cell death (parthanatos). J Neurochem 110, 687-696 (2009).
S. W. Yu, S. A. Andrabi, H. Wang, N. S. Kim, G. G. Poirier, T. M. Dawson, V. L. Dawson, Apoptosis-inducing factor mediates poly(ADP-ribose) (PAR) polymer-induced cell death. Proc Natl Acad Sci USA 103, 18314-18319 (2006).
J. P. Gagne, M. Isabelle, K. S. Lo, S. Bourassa, M. J. Hendzel, V. L. Dawson, T. M. Dawson, G. G. Poirier, Proteome-wide identification of poly(ADP-ribose) binding proteins and poly(ADP-ribose)-associated protein complexes. Nucleic Acids Res 36, 6959-6976 (2008).
P. L. Panzeter, B. Zweifel, M. Malanga, S. H. Waser, M. Richard, F. R. Althaus, Targeting of histone tails by poly(ADP-ribose). J Biol Chem 268, 17662-17664 (1993).
M. J. Eliasson, K. Sampei, A. S. Mandir, P. D. Hum, R. J. Traystman, J. Bao, A. Pieper, Z. Q. Wang, T. M. Dawson, S. H. Snyder, V. L. Dawson, Poly(ADP-ribose) polymerase gene disruption renders mice resistant to cerebral ischemia. Nat Med 3, 1089-1095 (1997).
J. P. Gagne, J. M. Hunter, B. Labrecque, B. Chabot, G. G. Poirier, A proteomic approach to the identification of heterogeneous nuclear ribonucleoproteins as a new family of poly(ADP-ribose)-binding proteins. Biochem J 371, 331-340 (2003).
H. Ye, C. Cande, N. C. Stephanou, S. Jiang, S. Gurbuxani, N. Larochette, E. Daugas, C. Gamido, G. Kroemer, H. Wu, DNA binding is required for the apoptogenic action of apoptosis inducing factor. Nat Struct Biol 9, 680-684 (2002).
M. J. Mate, M. Ortiz-Lombardia, B. Boitel, A. Haouz, D. Tello, S. A. Susin, J. Penninger, G. Kroemer, P. M. Alzari, The crystal structure of the mouse apoptosis-inducing factor AIF. Nat Struct Biol 9, 442-446 (2002).
M. D. Miramar, P. Costantini, L. Ravagnan, L. M. Saraiva, D. Haouzi, G. Brothers, J. M. Penninger, M. L. Peleato, G. Kroemer, S. A. Susin, NADH oxidase activity of mitochondrial apoptosis-inducing factor. J Biol Chem 276, 16391-16398 (2001).
J. A. Klein, C. M. Longo-Guess, M. P. Rossmann, K. L. Sebum, R. E. Hurd, W. N. Frankel, R. T. Bronson, S. L. Ackerman, The harlequin mouse mutation downregulates apoptosis-inducing factor. Nature 419, 367-374 (2002).
S. W. Yu, Y. Wang, D. S. Frydenlund, O. P. Ottersen, V. L. Dawson, T. M. Dawson, Outer mitochondrial membrane localization of apoptosis-inducing factor: mechanistic implications for release. ASN Neuro 1, pii: e00021. doi: 00010.01042/AN20090046 (2009).
M. Arundine, M. Tymianski, Molecular mechanisms of glutamate-dependent neurodegeneration in ischemia and traumatic brain injury. Cell Mol Lift Sci 61, 657-668 (2004).
S. W. Yu, Y. Wang, D. S. Frydenlund, O. P. Ottersen, V. L. Dawson, T. M. Dawson, Outer mitochondrial membrane localization of apoptosis-inducing factor: mechanistic implications for release. ASN Neuro 1, (2009).
D. Ahel, Z. Horejsi, N. Wiechens, S. E. Polo, E. Garcia-Wilson, I. Ahel, H. Flynn, M. Skehel, S. C. West, S. P. Jackson, T. Owen-Hughes, S. J. Boulton, Poly(ADP-ribose)-dependent regulation of DNA repair by the chromatin remodeling enzyme ALC1. Science 325, 1240-1243 (2009).
I. Ahel, D. Ahel, T. Matsusaka, A. J. Clark, J. Pines, S. J. Boulton, S. C. West, Poly(ADP-ribose)-binding zinc finger motifs in DNA repair/checkpoint proteins. Nature 451, 81-85 (2008).
P. Chang, M. Coughlin, T. J. Mitchison, Tankyrase-1 polymerization of poly(ADP-ribose) is required for spindle structure and function. Nat Cell Biol 7, 1133-1139 (2005).
A. J. Gottschalk, G. Timinszky, S. E. Kong, J. Jin, Y. Cai, S. K. Swanson, M. P. Washburn, L. Florens, A. G. Ladurner, J. W. Conaway, R. C. Conaway, Poly(ADP-ribosyl)ation directs recruitment and activation of an ATP-dependent chromatin remodeler. Proc Natl Acad Sci USA 106, 13770-13774 (2009).
V. Schreiber, F. Dantzer, J. C. Ame, G. de Murcia, Poly(ADP-ribose): novel functions for an old molecule. Nat Rev Mol Cell Biol 7, 517-528 (2006).
G. Timinszky, S. Till, P. O. Hassa, M. Hothorn, G. Kustatscher, B. Nijmeijer, J. Colombelli, M. Altmeyer, E. H. Stelzer, K. Scheffzek, M. O. Hottiger, A. G. Ladurner, A macrodomain-containing histone rearranges chromatin upon sensing PARP1 activation. Nat Struct Mol Biol 16, 923-929 (2009).
M. Stilmann, M. Hinz, S. C. Arslan, A. Zimmer, V. Schreiber, C. Scheidereit, A nuclear poly(ADP-ribose)-dependent signalosome confers DNA damage-induced IkappaB kinase activation. Mol Cell 36, 365-378 (2009).
G. M. Shah, S. H. Kaufmann, G. G. Poirier, Detection of poly(ADP-ribose) polymerase and its apoptosis-specific fragment by a nonisotopic activity-western blot technique. Anal Biochem 232, 251-254 (1995).
G. Brochu, G. M. Shah, G. G. Poirier, Purification of poly(ADP-ribose) glycohydrolase and detection of its isoforms by a zymogram following one- or two-dimensional electrophoresis. Anal Biochem 218, 265-272 (1994).
E. B. Affar, P. J. Duriez, R. G. Shah, E. Winstall, M. Germain, C. Boucher, S. Bourassa, J. B. Kirkland, G. G. Poirier, Immunological determination and size characterization of poly(ADP-ribose) synthesized in vitro and in vivo. Biochim Biophys Acta 1428, 137-146 (1999).
C. C. Kiehlbauch, N. Aboul-Ela, E. L. Jacobson, D. P. Ringer, M. K. Jacobson, High resolution fractionation and characterization of ADP-ribose polymers. Anal Biochem 208, 26-34 (1993).
V. L. Dawson, T. M. Dawson, D. A. Bartley, G. R. Uhl, S. H. Snyder, Mechanisms of nitric oxide-mediated neurotoxicity in primary brain cultures. J Neurosci 13, 2651-2661 (1993).
M. Gonzalez-Zulueta, L. M. Ensz, G. Mukhina, R. M. Lebovitz, R. M. Zwacka, J. F. Engelhardt, L. W. Oberley, V. L. Dawson, T. M. Dawson, Manganese superoxide dismutase protects nNOS neurons from NMDA and nitric oxide-mediated neurotoxicity. J Neurosci 18, 2040-2055 (1998).
B. M. Polster, K. W. Kinnally, G. Fiskum, BH3 death domain peptide induces cell type-selective mitochondrial outer membrane permeability. J Biol Chem 276, 37887-37894 (2001).
Y. A. Lazebnik, S. Cole, C. A. Cooke, W. G. Nelson, W. C. Earnshaw, Nuclear events of apoptosis in vitro in cell-free mitotic extracts: a model system for analysis of the active phase of apoptosis. J Cell Biol 123, 7-22 (1993).
H. Wang, S. W. Yu, D. W. Koh, J. Lew, C. Coombs, W. Bowers, H. J. Federoff, G. G. Poirier, T. M. Dawson, V. L. Dawson, Apoptosis-inducing factor substitutes for caspase executioners in NMDA-triggered excitotoxic neuronal death. J Neurosci 24, 10963-10973 (2004).
A. S. Mandir, M. F. Poitras, A. R. Berliner, W. J. Herring, D. B. Guastella, A. Feldman, G. G. Poirier, Z. Q. Wang, T. M. Dawson, V. L. Dawson, NMDA but not non-NMDA excitotoxicity is mediated by Poly(ADP-ribose) polymerase. J Neurosci 20, 8005-8011 (2000).
J. M. Pleschke, H. E. Kleczkowska, M. Strohm, F. R. Aithaus, Poly(ADP-ribose) binds to specific domains in DNA damage checkpoint proteins. J Biol Chem 275, 40974-40980 (2000).

BIOSEQUENCES
SEQ. ID NO. 1-Mouse PBM1
GGVAVLTGKKVVHLDVRGNMVKL
SEQ. ID NO. 2-Human PBM1
GGVAVLTGKKVVQLDVRDNMVKL
SEQ. ID NO. 3-Rat PBM1
GGVAVLTGKKVVHLDVRGNMVKL
SEQ. ID NO. 4-Chicken PBM1
GGVAVLSGKKVVHMDVRGNTVKL
SEQ. ID NO. 5-Mouse PBM2
FYDIKLGRRRVEHHDHAVVSGRL
SEQ. ID NO. 6-Human PBM2
FYDIKLGRRRVEHHDHAVVSGRL
SEQ. ID NO. 7-Rat PBM2
FYDIKLGRRRVEHHDHAVVSGRL
SEQ. ID NO. 8-Chicken PBM2
FYDIKLGRRRVEHHDHAVVSGRL
SEQ. ID NO. 9-Drosophila PBM2
FFDPLLGRRRVEHHDHSVVSGRL
SEQ. ID NO. 10-Mouse PBM3
LRDKVVVGIVLWNVFNRMPIARKIIK
SEQ. ID NO. 11-Human PBM3
LRDKVVVGIVLWNIFNRMPIARKIIK
SEQ. ID NO. 12-Rat PBM3
LRDKVVVGIVLWNVFNRMPIARKIIK
SEQ. ID NO. 13-Chicken PBM3
LRDKVVVGIVLWNIFNRMPIARKIIK
SEQ. ID NO. 14-Drosophila PBM3
LKNDKIVGILLWNLFNRIGLARTII
SEQ. ID NO. 15-Sm
LRDKVVVGIVLWNVFNRMPIARAIIK
SEQ. ID NO. 16-Dm
LRDKVVVGIVLWNVFNRMPIAAAIIK
SEQ. ID NO. 17-Tm
LRDKVVVGIVLWNVFNRMPIAAAIIA
SEQ. ID NO. 18-Qm
LRDKVVVGIVLWNVFNAMPIAAAIIA
SEQ. ID NO. 19-Dm2
LRDKVVVGIVLWNVFNRMPIALLIIK
SEQ. ID NO. 20-Dm3
LRDKVVVGIVLWNVFNRMPIALKIIL
SEQ. ID NO. 21-Dm4
LRDKVVVGIVLWNVFNRMPIARLIIL
SEQ. ID NO. 21-Tm2
LRDKVVVGIVLWNVFNRMPIALLIIL
SEQ. ID NO. 22-Human PBM1x
GGVAVLTGXXVVQLDVRDNMVKL
SEQ. ID NO. 23-Human PBM2x
FYDIXLGRXXVEXHDHAVVSGRL
SEQ. ID NO. 24-Human PBM3x
LRDKVVVGIVLWNIFNXMPIAXXIIX

1. A compound comprising a peptide having a sequence comprising any one of SEQ. ID NO. 15 through SEQ. ID NO. 24, wherein X represents an amino acid with the caveat that at least two X in the peptide is a hydrophobic amino acid selected from the group consisting of arginine (“A”) or leucine (“L”).

2. A polynucleotide including an encoding region that encodes the compound as recited in claim 1.

3. A recombinant vector which includes the polynucleotide according to claim 2.

4. A transformant having inserted therein the recombinant vector as recited in claim 3.

5. A method for producing a compound, which method includes the step of culturing the transformant of claim 4 above so as to induce the transformant to produce said compound.

6. A method for treating a disease or condition presenting unwanted parthanatos in target cells, comprising causing said target cells to express a mutated version of apoptosis inducing factor (“AIF”) which does not bind with Poly(ADP-ribose) (“PAR”).

7. The method as recited in claim 6, wherein said modified version includes a peptide having a sequence selected from the group consisting SEQ. ID NO. 15 through SEQ. ID NO. 24, wherein X represents an amino acid with the caveat that at least three X is a hydrophobic amino acid selected from the group consisting of arginine (“A”) or leucine (“L”).

8. The method as recited in claim 7, further comprising transfecting said target cells such that they express said mutated version of AIF.

9. The method as recited in claim 8, wherein said transfecting is done by infecting the cells with a transduced lentivirus.

10. The method according to claim 7, wherein said target cells are present in a human, and wherein said sequence is selected from the group consisting of SEQ. ID NO. 22, SEQ. ID NO. 23, and SEQ. ID NO. 24.

11. The method according to claim 6, wherein said disease or condition is a central nervous system (“CNS”) disease or condition selected from the group consisting of stroke, ischemia reperfusion injury of the CNS, postoperative CNS complications, traumatic brain injury, spinal cord injury, Parkinson's disease, Alzheimer's disease, multiple sclerosis, retinopathy, and macular degeneration.

12. The method according to claim 6, wherein said disease or condition is a neurodegenerative or neurologic disease or condition selected from the group consisting of Alexander's disease, Alper's disease, Alzheimer's disease, amyotrophic lateral sclerosis, ataxia telangiectasia, Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Sandhoff's disease, Schilder's disease, subacute combined degeneration of spinal cord secondary to pernicious anaemia, schizophrenia, spinocerebellar ataxia, spinal muscular atrophy, Steele-Richardson-Olszewski disease, and tabes dorsalis.

13. The method according to claim 12, wherein the disease or condition is Parkinson's disease.

14. The method according to claim 6, wherein said disease or condition is selected from the group consisting of diabetes, diabetes mellitus, diabetic retinopathy, diabetic endovascular disease, diabetic nephropathy, and diabetic neuropathy.

15. The method according to claim 6, wherein said disease or condition is a cardiovascular disease or condition selected from the group consisting of acute myocardial infarction, heart disease, cardiac allograft rejection, postoperative cardiac complications, myocarditis, heart failure, atherosclerosis, vascular hyporeactivity in sepsis, and circulatory shock.

16. The method according to claim 12, wherein the disease or condition is reperfusion injury, arthritis or colitis.

17. A method for treating a disease or medical condition in a mammal, said disease or medical condition being known to cause unwanted parthanatos in certain target cells of said mammal, said method comprising administering to said mammal a pharmaceutically effective amount of a PAR-AIF binding inhibitor drug, wherein said inhibitor drug following said administering:
(1) inhibits apoptosis inducing factor (“AIF”) from binding to Poly(ADP-ribose) (“PAR”) in said target cells substantially without interfering with non-PAR related cellular functions of AIF, or
(2) prevents AIF from releasing from the mitochondria membrane in response to nuclear PAR release.

18. The method according to claim 17, wherein said disease or medical condition is selected from the group consisting of a central nervous system (“CNS”) disease or condition, a diabetes related disease or condition, a reperfusion injury, and a cardiovascular disease or condition.

19. The method according to claim 17, wherein said mammal is a human, and said PAR-AIF binding inhibitor drug binds to a portion of human AIF that substantially includes a sequence selected from the group consisting of SEQ. ID NO. 2, SEQ. ID NO. 6, and SEQ. ID NO. 11.

20. A method for screening drug candidates for potential efficacy in humans to treat symptoms caused by a disease or medical condition that causes unwanted parthanatos in target cells, said method comprising testing each said drug candidate for the capability to prevent the release of apoptosis inducing factor (“AIF”) from the mitochondrial membrane in response to Poly(ADP-ribose) (“PAR”) binding in said target cells.

21. The method according to claim 20, wherein said testing further comprises:
inducing parthanatos in a control culture of said target cells,
inducing parthanatos in a test culture comprising said target cells treated with an amount of a particular drug candidate,
measuring cell death in said control culture and said test culture, and
designating said particular drug candidate as being potentially efficacious if the measured cell death in said test culture is lower than the measured cell death in the test culture by a statistically significant amount.

22. A method for screening drug candidates for potential efficacy in humans to treat symptoms caused by a disease or medical condition that causes unwanted parthanatos in target cells, said method comprising testing each said drug candidate for the capability to inhibit the binding of apoptosis inducing factor (“AIF”) to Poly(ADP-ribose) (“PAR”) in said target cells.

23. The method according to claim 22, wherein said testing comprises determining whether a given drug candidate binds to a portion of human AIF that substantially includes a sequence selected from the group consisting of SEQ. ID NO. 2, SEQ. ID NO. 6, and SEQ. ID NO. 11.

24. The method according to claim 22, wherein said testing further comprises:
inducing parthanatos in a control culture of said target cells,
inducing parthanatos in a test culture comprising said target cells treated with an amount of a particular drug candidate,
measuring cell death in said control culture and said test culture, and
designating said particular drug candidate as being potentially efficacious if the measured cell death in said test culture is lower than the measured cell death in the test culture by a statistically significant amount.

25. The method according to claim 21, wherein said testing further comprises determining when said drug candidate binds to a portion of human AIF that substantially includes a sequence selected from the group consisting of SEQ. ID NO. 2, SEQ. ID NO. 6, and SEQ. ID NO. 11.

26. A method for inhibiting the growth of a tumor in a mammal, said method comprising administering to said mammal a therapeutically effective amount of a drug that causes at least one of:
(1) target cells of said tumor to release nuclear Poly(ADP-ribose) (“PAR”) and thereby triggering parthanatos, or
(2) apoptosis inducing factor (“AIF”) release from the mitochondria of said target cells and thereby triggering parthanatos.

27. The method according to claim 26, wherein said target cells are cancerous.


SEQUENCE LISTING (25 sequences)

SEQ ID NO: 1 - protein, 23 aa, mouse
GGVAVLTGKKVVHLDVRGNMVKL

SEQ ID NO: 2 - protein, 23 aa, human
GGVAVLTGKKVVQLDVRDNMVKL

SEQ ID NO: 3 - protein, 23 aa, rat
GGVAVLTGKKVVHLDVRGNMVKL

SEQ ID NO: 4 - protein, 23 aa, chicken
GGVAVLSGKKVVHMDVRGNTVKL

SEQ ID NO: 5 - protein, 23 aa, mouse
FYDIKLGRRRVEHHDHAVVSGRL

SEQ ID NO: 6 - protein, 23 aa, human
FYDIKLGRRRVEHHDHAVVSGRL

SEQ ID NO: 7 - protein, 23 aa, rat
FYDIKLGRRRVEHHDHAVVSGRL

SEQ ID NO: 8 - protein, 23 aa, chicken
FYDIKLGRRRVEHHDHAVVSGRL

SEQ ID NO: 9 - protein, 23 aa, Drosophila
FFDPLLGRRRVEHHDHSVVSGRL

SEQ ID NO: 10 - protein, 26 aa, mouse
LRDKVVVGIVLWNVFNRMPIARKIIK

SEQ ID NO: 11 - protein, 26 aa, human
LRDKVVVGIVLWNIFNRMPIARKIIK

SEQ ID NO: 12 - protein, 26 aa, rat
LRDKVVVGIVLWNVFNRMPIARKIIK

SEQ ID NO: 13 - protein, 26 aa, chicken
LRDKVVVGIVLWNIFNRMPIARKIIK

SEQ ID NO: 14 - protein, 25 aa, Drosophila
LKNDKIVGILLWNLFNRIGLARTII

SEQ ID NO: 15 - protein, 26 aa, mouse
LRDKVVVGIVLWNVFNRMPIARAIIK

SEQ ID NO: 16 - protein, 26 aa, mouse
LRDKVVVGIVLWNVFNRMPIAAAIIK

SEQ ID NO: 17 - protein, 26 aa, mouse
LRDKVVVGIVLWNVFNRMPIAAAIIA

SEQ ID NO: 18 - protein, 26 aa, mouse
LRDKVVVGIVLWNVFNAMPIAAAIIA

SEQ ID NO: 19 - protein, 26 aa, mouse
LRDKVVVGIVLWNVFNRMPIALLIIK

SEQ ID NO: 20 - protein, 26 aa, mouse
LRDKVVVGIVLWNVFNRMPIALKIIL

SEQ ID NO: 21 - protein, 26 aa, mouse
LRDKVVVGIVLWNVFNRMPIARLIIL

SEQ ID NO: 22 - protein, 26 aa, mouse
LRDKVVVGIVLWNVFNRMPIALLIIL

SEQ ID NO: 23 - protein, 23 aa, human
  misc_feature (9)..(10): Xaa can be any naturally occurring amino acid
GGVAVLTGXXVVQLDVRDNMVKL

SEQ ID NO: 24 - protein, 23 aa, human
  misc_feature (5)..(5): Xaa can be any naturally occurring amino acid
FYDIXLGRXXVEXHDHAVVSGRL

SEQ ID NO: 25 - protein, 26 aa, human
  misc_feature (17)..(17): Xaa can be any naturally occurring amino acid
LRDKVVVGIVLWNIFNXMPIAXXIIX